SEQUENCE LISTING (6 sequences)

SEQ ID NO: 1 - protein, 18 aa, unidentified
MKLCILLAVVAFVGLSLG

SEQ ID NO: 2 - protein, 29 aa, synthetic construct
WSHPQFEKGGGSGGGSGGSSAWSHPQFEK

SEQ ID NO: 3 - protein, 11 aa, synthetic construct
GSGTAGGGSGS

SEQ ID NO: 4 - DNA, 2447 bp, Drosophila melanogaster
  source 1..2447: mol_type = genomic DNA
ctcctgataacgcaattgtattgtttatatagtttctaaagccgaacgagaatgcaatgtggcgatatcagttgctgataagcactggaaccctgcagtacacaacccataaccgacgatgactgccaccaaaaagcgtctgaagatcgttgcagccatctcgctgctcctcctgctcctggtctacctttaccgaatggccagcttctgcccaagcggaaaggtcgccgtttccgttcccggcgtcgaggaggttcaggccaaatggccgcccacggaaagtccgctacaaaggagccttcagatggcgtacgaggagcagagctctcttattcgagaacaaaaggccgaactccagcgcacaagggagaatttggccagattggaggaacaagttcgtagcctccagaccagcacaccgcgcaagtatccgaaggttaaatacctgaactataagaatcgcaaacgcatactaattaccggaggagctggcttcgtgggctcccacttggtcgacgatctgatggtccaggggcacgaggtcatcgtggtggacaatttctttacgggacgcaagcgaaatgtcgagcactggctggggcacgagaacttcgagctcatccaccacgacatagtgaatccgcttttcatcgagatcgatgagatctaccacttggcatcgccagcctcgccaccgcattacatgtacaatccggtgaagaccatcaagaccaacaccatgggcacgatcaatgtactgggtaaatatttataatatcaatcaaagaaaataaggattacacctaacgagtatatcttttttgaaaggtctggccaagcgcgtgatggccaaggttttgatagctagcacttcggaagtgtacggcgatcccacagtgcatccgcagccagaaacatactggggacatgtaaaccctattggaccgagagcctgttatgacgagggaaagcgtgtctccgagacgctgagctatgcgtatgccaaacaggtaatacctttagcaatatatatacttttttggataaccacattcctaagccagatcgaaaaaaaaacattaaactagctgagatccagaaatgtggcagatttttcctgtttcgatcgctagttgagcattaaatgatctcaattggaatgctattaattgatgttccgctcggttggctgacgcgagataagatttccaagcgcgagcactgaccacattttttgatcttggccagcaatctgggagtcaaattaattgctctttaaatgtatctgtatcgacaggagaaagtgcaagtccgagtggcgcgcatctttaacacttatggaccgagaatgcatatgaacgatggacgagtggtgtcaaactttatacttcaggcgctgaggaatgaaaccattacggtgtatggaaatggcaaacaaacgcgctcctttcagtatgtttcggatctggtggacggaatgatagccctaatggcatccaactacacacagccggtgaatttaggcaatcctgtggagcagacaatcggcgaatttgccgagatcatcaagaaactggtcggcgggcccagtgtgataaagcaatcgaaggccatggaagatgatccgcagcgcagaaaaccggacataactcgagcccgccagttgctccattgggagccaaaggttcccctagagactggacttcagcggaccatctcctacttccgcaacgaattggcgcgtagcgatcgctttcaggaaagctccaacaagtactttgatacacatacaccgtagaattaattgggcttagctctaattggtgatgatggaacgtctctagtctgcaaatgtaaatatgaccatcgttagcactaattgtaagccctcaaggaccacagaatacctttgtcatggttaattttttgaagttttgcgatatatcagcctatgtatggattaggaaatagtgatgtataaatgcgatcaaatgcactgaattctagaaaactaatttgaatttgtttaggaaaagaaaatccccaaagaatacagaacttagggagtatggaaacaacagttacaaacggtttattattcacggacgacattgctattcttttgaccgcatgtgtaccacatagacacagaatcactgggttacatgacacttagaaacagtataatccattgtattgtagaaccactatcgtattttaaaagtgcccacaaattcatacgttacatcagaaggttgcggcatatacatatatcagaaccagtgagaattgaacaaattattgaagatctgtgactcacaactttagacacataacacctatatgcgcctttaaccagtgtaagcacaccccgattctagaagataagtagggttttccagttgctgtagttggtagtccattaaaagagattttttctgcgaact

SEQ ID NO: 5 - RNA, 1326 nt, Drosophila melanogaster
  source 1..1326: mol_type = mRNA
atgactgccaccaaaaagcgtctgaagatcgttgcagccatctcgctgctcctcctgctcctggtctacctttaccgaatggccagcttctgcccaagcggaaaggtcgccgtttccgttcccggcgtcgaggaggttcaggccaaatggccgcccacggaaagtccgctacaaaggagccttcagatggcgtacgaggagcagagctctcttattcgagaacaaaaggccgaactccagcgcacaagggagaatttggccagattggaggaacaagttcgtagcctccagaccagcacaccgcgcaagtatccgaaggttaaatacctgaactataagaatcgcaaacgcatactaattaccggaggagctggcttcgtgggctcccacttggtcgacgatctgatggtccaggggcacgaggtcatcgtggtggacaatttctttacgggacgcaagcgaaatgtcgagcactggctggggcacgagaacttcgagctcatccaccacgacatagtgaatccgcttttcatcgagatcgatgagatctaccacttggcatcgccagcctcgccaccgcattacatgtacaatccggtgaagaccatcaagaccaacaccatgggcacgatcaatgtactgggtctggccaagcgcgtgatggccaaggttttgatagctagcacttcggaagtgtacggcgatcccacagtgcatccgcagccagaaacatactggggacatgtaaaccctattggaccgagagcctgttatgacgagggaaagcgtgtctccgagacgctgagctatgcgtatgccaaacaggagaaagtgcaagtccgagtggcgcgcatctttaacacttatggaccgagaatgcatatgaacgatggacgagtggtgtcaaactttatacttcaggcgctgaggaatgaaaccattacggtgtatggaaatggcaaacaaacgcgctcctttcagtatgtttcggatctggtggacggaatgatagccctaatggcatccaactacacacagccggtgaatttaggcaatcctgtggagcagacaatcggcgaatttgccgagatcatcaagaaactggtcggcgggcccagtgtgataaagcaatcgaaggccatggaagatgatccgcagcgcagaaaaccggacataactcgagcccgccagttgctccattgggagccaaaggttcccctagagactggacttcagcggaccatctcctacttccgcaacgaattggcgcgtagcgatcgctttcaggaaagctccaacaagtactttgatacacatacaccgtag

SEQ ID NO: 6 - protein, 441 aa, Drosophila melanogaster
MTATKKRLKIVAAISLLLLLLVYLYRMASFCPSGKVAVSVPGVEEVQAKWPPTESPLQRSLQMAYEEQSSLIREQKAELQRTRENLARLEEQVRSLQTSTPRKYPKVKYLNYKNRKRILITGGAGFVGSHLVDDLMVQGHEVIVVDNFFTGRKRNVEHWLGHENFELIHHDIVNPLFIEIDEIYHLASPASPPHYMYNPVKTIKTNTMGTINVLGLAKRVMAKVLIASTSEVYGDPTVHPQPETYWGHVNPIGPRACYDEGKRVSETLSYAYAKQEKVQVRVARIFNTYGPRMHMNDGRVVSNFILQALRNETITVYGNGKQTRSFQYVSDLVDGMIALMASNYTQPVNLGNPVEQTIGEFAEIIKKLVGGPSVIKQSKAMEDDPQRRKPDITRARQLLHWEPKVPLETGLQRTISYFRNELARSDRFQESSNKYFDTHTP

(19) United States
(12) Patent Application Publication
(10) Pub. No.: US 2026/0184752 A1
(43) Pub. Date: Jul. 2, 2026

(54) RECOMBINANT PRODUCTION OF PROTEIN HAVING XYLOSYLATED N-GLYCANS

(71) Applicant: EXPRES2ION BIOTECHNOLOGIES APS, Hørsholm (DK)

(72) Inventors: Magdalena Skrzypczak, Hørsholm (DK); Ida Busch Nielsen, Hørsholm (DK); Stine Broch Clemmensen, Hørsholm (DK); Willem Adriaan De Jongh, Hørsholm (DK)

(73) Assignee: EXPRES2ION BIOTECHNOLOGIES APS, Hørsholm (DK)

(21) Appl. No.: 19/130,725

(22) PCT Filed: Nov. 17, 2023
(86) PCT No.: PCT/EP2023/082208

§ 371 Date: May 16, 2025

(30) Foreign Application Priority Data
Nov. 17, 2022 (EP) 22208158.0

(51) Int. Cl. C07K 14/505; C07K 14/82; C12N 1/16; C12N 5/07; C12N 5/071; C12N 5/10; C12N 9/10; C12N 9/88; C12P 21/00

(52) U.S. Cl. CPC C07K 14/505; C07K 14/82; C12N 1/16; C12N 5/0601; C12N 5/0682; C12N 5/0686; C12N 5/10; C12N 9/1077; C12N 9/88; C12P 21/005; C12Y 204/02038; C12Y 401/01035; C12N 2510/02

(57) ABSTRACT

Provided are non-plant polypeptides or proteins comprising xylosylated N-linked glycans that comprise β1,2-xylose, wherein at least 25% of the individual protein species comprise xylosylated N-glycans, compositions comprising the non-plant proteins, cells engineered to produce the polypeptide or proteins, and methods of their production and use.

### FIELD OF THE INVENTION

The present invention relates to the field of active immunization. In particular, the present invention provides immunization antigens that have been glycoengineered to include non-native glycosylation patterns with a view to enhance their properties as antigens for use in areas such as vaccination and antibody production. The invention also pertains to means and methods for producing the glyco-modified antigens as well as methods and uses of the glyco-modified antigens.

### BACKGROUND OF THE INVENTION

Protein therapeutics such as monoclonal antibodies (mAbs), peptides and recombinant proteins, represent a large group of developing products in the biopharmaceutical industry. The majority of biological FDA-approved products are recombinant glycoproteins, which are used in treatment against a range of diseases, such as metabolic disorders, autoimmune diseases, and cancer. These products are produced in a wide variety of platforms, such as mammalian expression systems, including CHO and human cell lines, and non-mammalian expression systems, such as bacterial, yeast, plant and insect.

The most appropriate expression system for a specific therapeutic protein depends on the particular protein to be expressed and the intended usage. In the past, proteins with therapeutic capabilities were derived directly from the source, for example humans or pigs. Examples hereof are insulin, which was derived from pancreatic tissue and albumin derived from blood plasma. However, assuring reproducibility, purity, and safety was difficult with the emergence of genetic engineering technology this led to the development of recombinant expression systems for protein production.

Different expression systems have different advantages, but certain therapeutics have glycosylation requirements that currently mean that primarily CHO and other mammalian systems can be used for their production. Other expression systems have advantages like speed and ability to produce difficult proteins.

Glycosylation is generally important to consider when producing vaccine antigens. Especially N-linked glycans are important, as they influence glycoprotein half-life, dictate migration, ensure protein stability, and mediate cell signalling.

The most common expression systems and their glycosylation characteristics are described in the following.

### Bacteria as an Expression System

In 1982 the first recombinant biopharmaceutical was approved. This was insulin (by Eli Lilly & Co.'s Humulin®) and it was produced in Escherichia coli. E. coli has since been used to produce commercially approved non-glycosylated therapeutic proteins, such as cytokines, and monoclonal antibodies and enzymes. Bacteria generally do not glycosylate proteins, as they lack glycosylation machinery. Due to the inability to add N-glycans to proteins, bacteria have a limitation in production compared with more complex hosts for proteins that require post-translational modifications (PTMs). However, the bacterium Campylobacter jejuni has exhibited a glycosylation machinery, which has successfully been transferred to E. coli. Although this is highly relevant for recombinant protein production, additional optimizations to establish a cost-effective process are still needed.

### Yeast as an Expression System

Yeast-based systems have been extensively used for recombinant protein expression. Yeast and filamentous fungi offer numerous advantages as recombinant protein expression systems when compared with mammalian cell culture, including high recombinant protein titers, short fermentation times, and the ability to grow in chemically defined media. Saccharomyces cerevisiae is the expression system for approximately 20% of all biopharmaceuticals, including insulin, hepatitis vaccines, and human serum albumin. Yeasts can be grown in industrial scale and they represent very robust expression, capable of folding proteins and secreting these into the medium. Furthermore, they also present well characterized N-glycosylation, which is often hyper-mannosylation. To produce better drugs many efforts have been made to humanize the N-glycosylation in yeasts, and in 2006 Hamilton et al managed to construct a Pichia pastoris cell line that adds 90.5% of double-sialylated N-glycan structures on purified erythropoietin (EPO).

### Plant Cells as an Expression System

Plant cells can be cultured in basal culture medium and are easily scaled up. Plant cells do not contain endotoxins like E. coli and they do not represent the same disadvantages as recombinant protein production in whole plants. Plant cells show greater similarity to human N-glycans than yeast does. However, plant cells are also known to express α1,3-fucose and β1,2-xylose, which both are considered immunogenic to the human immune system. In 2012 the first plant-based therapeutic was approved by the FDA. Elelyso (ProTalix BioTherapeutics), which is made in carrot cells and carry α1,3-fucose and β1,2-xylose, is intended for patients suffering from the lysosomal storage disease known as Gaucher disease. These individuals lack the enzyme glucocerebrosidase and previous treatment for this condition has been through administration of recombinant glucocerebrosidase produced from mammalian cells. As this production in mammalian cells is relatively expensive, efforts were put into producing glucocerebrosidase in a cheaper system. Remarkably, the plant-produced glucocerebrosidase does not seem to cause adverse immune reactions in humans.

Another example is SARS-CoV-2 virus vaccine candidate of the company Medicago. The vaccine is produced in Nicotiana benthamiana that has completed Phase III clinical trials and did not report adverse reactions in the subjects (C. Dubé et al., “Broad neutralization against SARS-CoV-2 variants induced by ancestral and B.1.351 AS03-Adjuvanted recombinant Plant-Derived Virus-Like particle vaccines,” Vaccine, vol. 40, no. 30, pp. 4017-4025, 2022, doi: 10.1016/j.vaccine.2022.05.046 and K. J. Hager et al., “Efficacy and Safety of a Recombinant Plant-Based Adjuvanted Covid-19 Vaccine,” N. Engl. J. Med., vol. 386, no. 22, pp. 2084-2096, 2022, doi: 10.1056/nejmoa2201300).

Although Medicago did not provide information on the glycosylation of their vaccine, Balieu et al. have produced SARS-CoV-2 Spike protein in N. benthamiana and analyzed its glycosylation and the predominant forms found were the ones that can be seen in FIG. 1 at the bottom row of plant glycan examples, with F(3)A2Xyl being the prevalent N-glycan.

### Mammalian Cells as an Expression System

More than 50% of therapeutic proteins available on the market are produced using mammalian cells. Generally, mammalian expression systems are preferred for manufacture of biopharmaceuticals that are large and complex proteins and which require post translational modifications (PTMs, most notably glycosylation) as these usually are relatively similar to proteins produced in humans. Moreover, in the case of mammalian cell lines, and animal cell lines in general, most proteins can be secreted directly into the growth medium, which is advantageous compared with bacteria/prokaryotes, where cell lysis is needed to extract protein and potential subsequent refolding of the protein. The most common mammalian (non-human) cell lines used for therapeutic protein production include murine myeloma cells (NS0 and Sp2/0), Chinese hamster ovary (CHO) cells, and baby hamster kidney (BHK21) cells. However, these non-human mammalian cell lines also have disadvantages. They produce glycosylation that is not expressed in humans, more specifically galactose-α1,3-galactose (α-gal) and N-glycolylneuraminic acid (Neu5Gc). Antibodies against both of these N-glycans are found in human circulation, therefore therapeutic drugs are screened during cell line development and production for an acceptable glycan profile. Glycan profiles are considered a critical quality parameter for therapeutic proteins.

### Insect Cells as an Expression System

Insect cells are easily cultured and can secrete correctly folded and posttranslationally modified proteins into the medium. The N-glycans in insect cells are, like plant N-glycans, comparable to human structures, but they are generally simpler. Most proteins produced in insect cell lines carry M3 or F(6)M3 and to some degree also high-mannose structures. The baculovirus expression system (BEVS) is the most common insect expression system and is used for many recombinant expression purposes. This insect cell based expression platform has successfully been used to produce vaccine antigens and virus-like particles. Until now, Cervarix® (GlaxoSmithKline) and FluBlok® (Protein Sciences) have been approved as vaccines by the FDA. Regulatory authorities have also approved Provenge® (Dendreon) which contains an Sf21 cell line produced protein as a component of the autologous prostate-cancer therapy product. The predominant N-glycan form found in commonly used insect cells is a short, paucimannosidic form (trimannosyl), with or without core α1,6-fucose. The Spodoptera frugiperda 9 (Sf9) insect cell line has been glyco-engineered to produce more complex N-glycosylation. There are, however, still relatively high levels of F(6)M3 left as well as intermediate glycan structures.

Some insect cells, such as the Trichoplusia ni derived High Five™ and Tni PRO™ cells, glycosylate in a similar M3 structure as Sf9 and Drosophila Schneider 2 (S2) cells, but with an immunogenic α1,3-linked fucose rather than a α1,6-linked as the Sf9 and S2 cells. Efforts have been made to remove fucosylation on proteins expressed in Sf9, High Five™ and Tni PRO™. The approach was not to directly target the genes responsible for the attachment of core α1,3- and α1,6-fucose, fut11/12 and FucT6 respectively, but instead targeting both α1,3- and α1,6-linked fucose at the same time by inserting a gene for an enzyme that consumes the immediate precursor to GDP-L-fucose to produce GDP-D-rhamnose, which would remove any substrate for fucose addition. This was successful, however, Mabashi-Asazuma et al. (2014), Glycobiology 24:325-340, saw issues with long-term stability of the cell lines.

The S2 insect cell line was originally established in 1971 by Imogene Schneider. Since then, around 100 Drosophila cell lines have been obtained out of which 12 are easily cultivated. However, the primary cell lines being used for recombinant protein production are two of the original Schneider cell lines: Schneider's 2 (S2) and 3 (S3). Both S2 and S3 can be genetically modified to express recombinant proteins independent of viral infection, unlike BEVS. However, only S2 cells have been used to produce vaccine antigens for clinical trials. Stably transfected S2 cells can grow at high cell densities (up to 50×10⁶ cells/mL) in suspension and S2 based production processes are scalable. It is well established that S2 cell recombinant proteins carry pauci-mannosidic glycans and often also attach core α1,6-fucose. In addition, the present inventors have also detected small amounts of high-mannose structures and A1.

The two most prevalent N-linked glycan structures found on protein secreted from S2 and Sf9 cells are M3 and F(6)M3. In the High Five™ cell line further two structures are also found, the immunogenic F(3)M3 and F(3) F(6)M3.

Xylose is not a common component of N-glycans in mammals or insect cells but occurs naturally in plants and helminth. Species specific glycosylation can impose challenges for production of therapeutic glycoproteins but at the same time opens possibility to modulate immune responses to vaccines.

To summarize, the different expression systems discussed above can be summarized as follows:

| | Expression System | | | | |
|---|---|---|---|---|---|
| | | | Plant cell | Insect cell | Mammalian cell |
| | Bacteria | Yeast | culture | culture | culture |
| Desired characteristics | | | | | |
| Cell growth | Rapid | Rapid | Slow | Slow | Slow |
| Complexity of growth medium | Minimum | Minimum | Complex | Complex | Complex |
| Cost of growth medium | Low | Low | Low | High | High |
| Expression level | High | Low to high | Low to high | Low to high | Low to moderate |
| Extracellular expression | Secretion to | Secretion to | Secretion to | Secretion to | Secretion to |
| | periplasm | medium | medium | medium | medium |
| Post tanslational modifications | | | | | |
| Protein folding | Refolding usually | Re folding may be | Proper folding | Proper folding | Proper folding |
| | required | required | | | |
| N-linked glycosylation | No | Hyper-mannose | Simple, no sialic | Simple, | Complex |
| | | | acid, but xylose | no sialic acid | |
| | | | and 1,3-fucose | | |
| O-linked glycosylation | No | Yes | Yes | Yes | Yes |
| Phosphorylation | No | Yes | Yes | Yes | Yes |
| Acetylation | No | Yes | Yes | Yes | Yes |
| Acylation | No | Yes | Yes | Yes | Yes |
| γ-Carboxylation | No | No | No | No | Yes |

The most common N-glycosilation in humans, insects and plants is summarised in FIG. 1. Fucose present on N-glycans can be α1,6-linked and/or α1,3-linked to the core GlcNAc or α1,4-linked to the GlcNAc preceding Gal (only in plants). 10

### Glycosylation and the Immune System

Generally, glycosylation of proteins plays an important role in various parts of vertebrate immune systems and is one of the most common post-translation modifications (PTMs).

Most N-glycans are comprised of sugar chains of N-acetylglucosamine (GlcNAc), mannose (Man), galactose (Gal), fucose (Fuc) and sialic acids (Sia) and differ in complexity different organisms.

Antibodies, or immunoglobulins (Ig), are glycoproteins, which are produced by the immune system to target foreign invading pathogens. Igs consist of a variable antigen-binding (Fab) fragment and a constant (Fc) fragment.

Variable Fab regions bind to high diverse molecular structures in proteins, carbohydrate and lipids. Antibodies can exist in a secreted form or as membrane-bound. There are five antibody isotypes. IgA is found in mucosal areas, such as the gut, respiratory tract and urogenital tract, saliva, tears and breast milk. IgD is an antigen receptor on B-cells that have not yet been exposed to antigens. IgE acts as a receptor on the surface of mast cells and basophils and triggers histamine release from these upon cross-binding to antigens; biologically, this action protects against parasitic worms but the reaction is also involved in type I allergy. IgG consists of four different isotypes and is the major antibody involved in immunity against invading pathogens. IgM is expressed on the surface of B cells as a monomer, but also in a secreted form as a di- or pentamer, which eliminates pathogens in the early stages of the B-cell mediated humoral response before sufficient levels of IgG are reached. Core fucose on the glycan structure limits the IgG binding to the IgG Fc receptor, which results in decreased antibody-dependent cell-mediated cytotoxicity.

Antibodies are produced by the adaptive immune system, more specifically by B cells. B cells mature in the bone marrow and on release, each expresses a unique antigen-binding receptor on its membrane. When a naïve B-cell first encounters the antigen that matches its membrane-bound antibody, the binding of the antigen to the antibody (in a process the normally requires concurrent stimulation from T helper lymphocytes that recognize processed antigen presented by the B-cell on its surface) causes the B-cell to divide rapidly into memory B-cells and effector B-cells. The memory B-cells have longer life span than their parent B-cell, and they continue to express membrane-bound antibody similar to their parent B-cell. Effector cells produce the antibody in a secreted form. Effector cells only survive for a few days; however, they secrete considerable amounts of antibodies. Secreted antibodies are the major effector of humoral immunity. Some antibodies play their role simply through the binding to the target epitopes to block or induce signal transduction, whereas other antibodies bind the antigen and then recruit circulating lymphoid and myeloid cells to kill the invading pathogen by antibody-mediated effector functions (i.e., complement-dependent cytotoxicity, antibody-dependent cell-mediated cytotoxicity, and antibody-dependent cellular phagocytosis).

Dendritic cells (DCs) are a primary link between the innate and the adaptive immune system in mammals. Their primary function is to present digested antigens to T cells. DCs are found in tissues that are in contact with the environment, such as the skin, inner linings of nose, lungs, stomach and intestines. Once a DC is activated, it will migrate to the lymph node and interact with B and T cells. This process shapes the adaptive immune response.

The immature DCs are constantly analyzing their surrounding environment for pathogens via their pattern recognition receptors (PRRs), such as Toll-like receptors (TLRs). These recognize specific repetitive structures found on pathogens. Immature dendritic cells phagocytize pathogens and degrade these into peptides and present them on their cell surface during maturation. The surface presentation is carried out by major histocompatibility complex (MHC) molecules, which present the peptides to T-cells. During maturation, the DCs up-regulate surface receptors, such as CD80, CD86, and CD40 that greatly contribute to T-cell activation. In turn, activated T-cells aid in the full maturation of B-cells and antibody production. DCs carry certain C-type lectin receptors (CLRs) on their surface, which help instruct the DCs as when to induce immune tolerance rather than an immune reaction. Examples of these C-type lectins are the mannose receptor (MR, CD206) and Dendritic Cell-Specific Intercellular adhesion molecule-3-Grabbing Non-integrin (DC-SIGN, CD209). Blood contains monocytes, which can be matured into DCs in vitro.

Innate immune responses are often initiated by macrophage lectins recognizing microbial glycans, which results in phagocytosis. Circulating lectins, such as serum mannose-binding lectin (MBL) and ficolins, bind to pathogen cell surfaces, hereby activating the complement cascade. When immune cells bind to glycans it can also activate intracellular signalling that either triggers or suppresses cellular responses. For example, binding of trehalose dimycolate, a glycolipid found in the cell wall of Mycobacterium tuberculosis by the macrophage C-type lectin Mincle, induces a signalling pathway that causes the macrophage to secrete pro-inflammatory cytokines. However, glycans can also have the opposite effect. For example, the B-lymphocyte carries a lectin called CD22, that when bound to α2,6-linked sialic acis initiates signalling that inhibits activation to prevent self-reactivity. Interestingly, the α2,6-linked sialic acid is also the gateway for the human influenza virus to enter human cells. The lectin of the virus, also called the hemagglutinin, facilitates binding to the host cell membrane and entry inside the cell. This interaction is highly specific. The human influenza virus recognizes α2,6-linked sialic acid and the bird influenza virus recognizes only α2,3-linked sialic acid.

The mannose receptor (MR), or Cluster of Differentiation 206 (CD206) is a C-type lectin, which is found on the surface of macrophages and dendritic cells. The MR has 8 recognition domains, which recognize terminal mannose, GlcNAc and fucose residues on glycans carried by proteins. MR has higher affinity towards branched mannose structures than linear ones, and preferably pauci-mannose structures. The MR plays a role in antigen uptake and presentation by immature DCs in the adaptive immune system. Upon binding, MR ensures delivery of the bound antigen to the early endosomes, and afterwards to the lysosomes. Here the antigen is degraded and presented on MHC class II molecules, which stimulates and polarizes the adaptive immune response.

Dendritic Cell-Specific Intercellular adhesion molecule-3-Grabbing Non-integrin (DC-SIGN) is, like MR, also a C-type lectin, which is found on the surface of macrophages and dendritic cells. This receptor also recognizes mannose, albeit DC-SIGN has higher specificity towards high-mannose, preferably M9, structures than pauci-mannose structures. DC-SIGN has been shown to bind relatively weak to F(6)M3 and to not bind F(3)M3 at all. DC-SIGN only encompasses one recognition site, however, it forms tetramers with other DC-SIGNs on the DC surface. Once DC-SIGN binds to a glycan or a microorganism it delivers the bound components to late endosomes or lysosomes, where they are destined for degradation. The degraded antigens are presented on MHC class II for T cell presentation. In specific cases it appears that both MR and DC-SIGN deliver antigens to MHC class I molecules.

Mannose Binding Lectin (MBL) is a secreted C-type lectin found in circulation, which recognizes mannose structures. Like the membrane bound MR and DC-SIGN, the recognition is not entirely specific, and MBL binds with higher specificity to high-mannose also recognizes fucose and GlcNAc. MBL encompasses a single receptor, and forms a trimer as a basic unit. When six trimmers aggregate a very strong binding is seen. In contrast to MR and DC-SIGN, MBL is capable of activating the innate immune system. Upon binding to a microorganism or antigen carrying mannose, the MBL activates the complement by the lectin pathway, followed by opsonization and phagocytosis.

Additionally, studies conducted by Sandig et al. (“Engineering of CHO Cells for the Production of Recombinant Glycoprotein Vaccines with Xylosylated N-glycans,” Bioengineering, vol. 4, no. 38, pp. 1-12, 2017, doi: 10.3390/bioengineering4020038) have shown that introducing xylose on N-glycans can positively influence potency of recombinant glycoprotein-based vaccines by increasing their immunogenicity. Recognition of pathogens is essential for the stimulation of T cell differentiation. Dendritic cells express different types of pattern recognition receptors (PRRs) that bind to structures present on pathogens, called pathogen-associated molecular patterns (PAMPs). Binding leads to antigen uptake, cytokine secretion and T cell differentiation. A particular pathogen can have a number of PAMPs that can simultaneously bind to dendritic cells and shape the immune response in a particular way. The role of non-human N-glycan structures in modulating the immune system is not well studied yet, but great focus lies on C-type lectin receptors (CLRs) present on dendritic cells that recognize carbohydrates structures from pathogen. For instance, Sandig et al. have shown that xylosylated F-protein of human respiratory syncytial virus produced in glycoengineered CHO cells has shown more favourable cytokine profile in comparison to the wild type, unxylosylated vaccine in human artificial lymph node reactor, and concluded that addition of xylose had clear adjuvant effects. On the other hand, Brzezicka et al. (“Influence of Core β-1,2-Xylosylation on Glycoprotein Recognition by Murine C-type Lectin Receptors and Its Impact on Dendritic Cell Targeting,” ACS Chem. Biol., vol. 11, no. 8, pp. 2347-2356, August 2016, doi: 10.1021/acschembio.6b00265) have screened biantennary GlcNAc N-glycans (G0) with and without xylose and found that the unxylosylated glycans lead to higher dendritic cell uptake and had higher affinity to several CLRs.

Larsen et al. (“Engineering mammalian cells to produce plant-specific N-glycosylation on proteins,” Glycobiology, vol. 30, no. 8, pp. 528-538, August 2020, doi: 10.1093/glycob/cwaa009) as well as Sandig et al. have shown that expression of a single gene of plant xylosyltransferase from Arabidopsis thaliana or Nicotiana tabacum is sufficient to obtain xylosylated N-glycans in CHO cells. This shows that mammalian cells have an intrinsic metabolic pathway for xylose and UDP-xylose synthesis.

WO 2020/144358 provides glycol-engineered antigens for use in immunization technology, including vaccination, which have improved antigenic/immunogenic properties compared to their non-modified counterparts.

To conclude, there is a continued need to provide recombinant proteins having engineered glycosylation designed for particular purposes, in particular when the purpose relates to immunotherapy and vaccination. It is also of value to overcome limitations in the native S2 glycosylation machinery, since this can lead to new and powerful expression systems for industry use, which would enable preparation of new drugs.

### OBJECT OF THE INVENTION

It is an object of embodiments of the invention to provide glyco-engineered antigens for use in immunization technology, including vaccination, which have improved antigenic/immunogenic properties compared to their non-modified counterparts. It is further an object of other embodiments of the invention to provide means and methods for preparation of the glyco-engineered antigens and also to provide methods and uses that employ the glyco-engineered antigens.

### SUMMARY OF THE INVENTION

It has been found by the present inventor(s) that a Drosophila S2 cell line produces xylosylated N-glycans by stably expressing β1,2-xylosyltransferase from Arabidopsis thaliana. Further, is has been found that xylosylated proteins produced from such cell lines are superior inducers of antibody response.

So, in a first aspect the present invention relates to a non-plant polypeptide or protein comprising N-linked glycans that comprise β1,2-xylose.

In a second aspect, the present invention relates to an immunogenic composition comprising a polypeptide of the first aspect of the invention in admixture with at least one immunological adjuvant and optionally a pharmaceutically acceptable carrier and/or diluent and/or excipient.

In a third aspect, the present invention relates to a method for inducing or enhancing a specific immune response in an animal, such as a human being, the method comprising at least one immunization of the animal with an effective amount of the protein or polypeptide of the first aspect of the invention or the composition of the second aspect of the invention.

In a fourth aspect, the invention relates to a genetically modified non-plant eukaryotic cell, such as a mammalian cell or an insect cell or a fungal cell such as a yeast, which comprises at least one heterologous polynucleotide sequence encoding and expressing β1,2-xylosyltransferase, wherein the cell is capable of producing N-glycosylated protein carrying β1,2-xylose, and where the heterologous polynucleotide sequence preferably is the XylT gene from Arabidobsis thaliana, or an equivalent polynucleotide encoding a plant β1,2-xylosyltransferase. This aspect also relates to a cell line, such as a clonal cell line, comprising a cell of the 4^{th} aspect.

Finally, in a fifth aspect the invention relates to a method for producing an N-glycosylated polypeptide or protein carrying β1,2-xylose, the method comprising culturing a cell according to the forth aspect, wherein the cell line expresses a polynucleotide encoding the amino acid sequence of the N-glycosylated polypeptide or protein, and subsequently isolating the N-glycosylated polypeptide or protein from the culture mixture, and optionally subjecting the N-glycosylated polypeptide or protein to further purification.

### LEGEND TO FIGURES

FIG. 1 shows the most common N-glycosylation in humans, insects, and plants. Fucose present on N-glycans can be α1,6-linked and/or α1,3-linked to the core GlcNAc or α1,4-linked to the GlcNAc preceding Gal (only in plants).

FIG. 2 shows the construct design of a vaccine antigen comprising part of Spike 1 protein of SARS-CoV-2.

FIG. 3 shows examples of structures and their nomenclature of N-linked glycans. Dark square: N-acetylglucosamine (GlcNAc), light grey circle: mannose, triangle: fucose, light grey circle: galactose, diamond: sialic acid

FIG. 4 shows the prevalent N-glycan forms in wild type S2 cells and S2 cells expressing plant β1,2-xylosyltransferase.

FIG. 5a-5e shows the secretome of three clones of the S2-xyIT cell line (named S2-Xyl clone 6, S2-Xyl clone 7 and S2-Xyl clone 8) were further analyzed by LS-MS to obtain detailed glycan profiles and were compared with the wild type glycoprofile. FIG. 5a shows the peak annotations for FIGS. 5b, 5c and 5d. FIG. 5b shows the glycoprofile of secretome of S2-WT (LC-MS, 2AB label). FIG. 5c shows the N-glycoprofile of secretome of S2-XylTclone 6 (LC-MS, Rapi Fluor label). FIG. 5d shows the N-glycoprofile of secretome of S2-XylT-clone 7 (LC-MS, Rapi Fluor label). FIG. 5e shows the N-glycoprofile of secretome of S2-XylT-clone 8 (LC-MS, Rapi Fluor label).

FIG. 6 shows the comparison of N-glycosylation in wild type S2 cells and three S2-Xyl clones on secretome level, relative percentage of released N-glycans analyzed by LC-MS-M3: paucimannose; F(6)M3: paucimannose with core α1,6-fucose; HM: high mannose; A1: paucimannose with one terminal GlcNAc; F(6)A1: paucimannose with one terminal GlcNAc and core α1,6-fucose; Xyl containing: mainly M3Xyl and F(6)M3Xyl.

FIG. 7 (A-D) shows the comparison of N-glycosylation of recombinant human erythropoietin expressed in S2-WT cells (A) and in S2-Xyl clone 8 (B). C shows the N-glycan structures for annotations expressed. D shows the comparison of relative percentages of N-glycans from hEPO expressed in S2-WT and S2-Xyl clone 8. M3: paucimannose; F(6)M3: paucimannose with core 1,6-fucose; HM: high mannose; A1: one terminal GlcNAc; F(6)A1: one terminal GlcNAc and 1,6-fucose; Xyl containing: F(6)M3X and F(6)A1X.

FIG. 8 shows the relative percentages of N-glycans on RBD expressed in S2-WT and S2-Xyl clone 8.

FIG. 9a-c shows the RBD of Spike 1 protein of SARS-CoV-2 was produced in S2-WT and S2-Xyl. Released N-glycans of (9A) RBD-WT and (9B) S2-Xyl were analyzed by LC-MS. (9C) Comparison of relative percentages of the glycans from RBD-WT and RBD-Xyl. M3: paucimannose; F(6)M3: paucimannose with core 1,6-fucose; HM: high mannose; A1: one terminal GlcNAc; F(6)A1: one terminal GlcNAc and 1,6-fucose; M3Xyl: xylosylated paucimannose; F(6)M3Xyl: xylosylated paucimannose with core 1,6-fucose.

FIG. 10 shows the reduced vaccines components seen on SDS-PAGE. M: Marker, Lane 1: (RBD-WT)-CVLP; Lane 2:: (RBD-Xyl)-cVLP, for Lane 1 and Lane 2 conjugated antigen-cVLP 60 kDa (upper band) and uncoupled cVLP 16.5 kDa (bottom band); Lane 3: Unconjugated CVLPs (16.5 kDa), Lane 4: RBD-WT (44 kDa); Lane 5: RBD-Xyl (44 kDa).

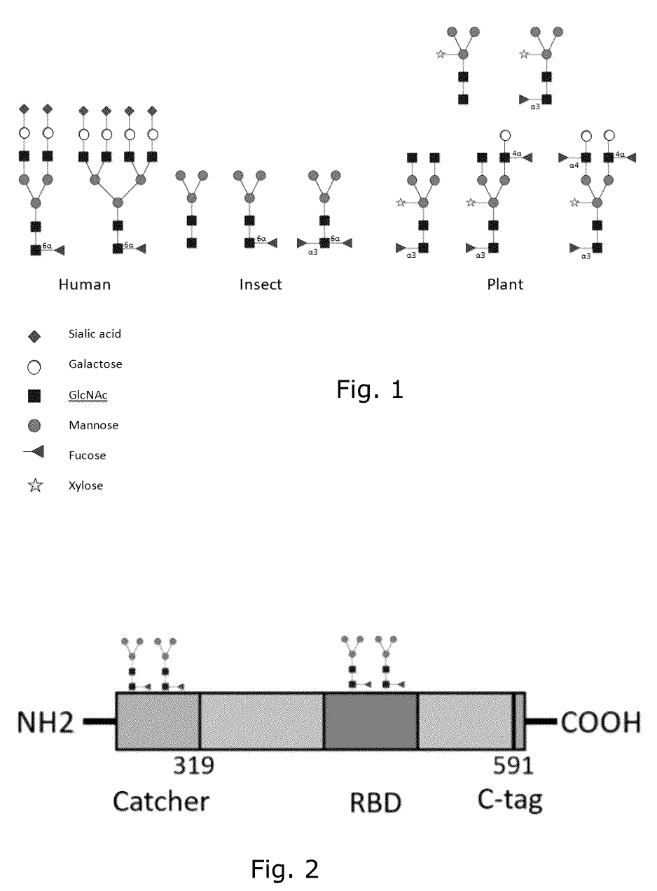

FIG. 11 shows Total IgG titers and IgG subclasses titers measured towards HEK293 produced Spike 1 protein.
A: Total anti-Spike IgG dilution curve detected from 1^{st} bleed of mice vaccinated with four vaccines
B: Total anti-Spike IgG dilution curve from 2^{nd} bleed of mice vaccinated with four vaccines.
C: Total IgG anti-Spike presented as AUCs of pre-bleed, 1^{st} bleed and 2^{nd} bleed samples.
D-G: AUCs for IgG subclasses from 2^{nd} bleed samples. For AUCs, each dot represents one mouse. For each graph, geometric mean with geometric SD is shown. Statistical significance was based on two-tailed, non-parametric Mann-Whitney test. Not significant (ns) when p>0.05; * when p≤0.05; ** when p<0.01.

FIG. 12 shows total anti-Spike 1 IgG dilution curves.
(A) In 1^{st} bleed.
(B) In 2^{nd} bleed.

FIG. 13 shows mass spectra of released N-glycans of secreted proteins of the S2-XylT-clone 8 cell line. “X” denotes an unannotated glycan structure.

FIG. 14 shows mass spectra of released N-glycans of secreted proteins of S2-XylT-UXS S2 cell line. “X” denotes an unannotated glycan structure. Arrows indicate apparent changes (lower or higher) in peak areas in S2-XylT-UXS in relation to peak areas in FIG. 13 for S2-XylT clone 8.

FIG. 15 shows Anti tag Western blot of CMV Gb variants expressed in WT, S2-XylT-clone 8 and S2-XylT-UXS cell lines.

Samples from transient transfection; samples of all secreted proteins (not purified protein).

FIG. 16 shows a depiction of an immunization scheme and blood sampling schedule.

### DETAILED DISCLOSURE OF THE INVENTION

### Definitions

The term “polypeptide” is in the present context intended to mean both short peptides of from 2 to 10 amino acid residues, oligopeptides of from 11 to 100 amino acid residues, and polypeptides of more than 100 amino acid residues. Furthermore, the term is typically also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups. In the present application, polypeptides and proteins are all glycosylated.

A “non-plant” polypeptide or protein denotes a protein, the amino acid sequence of which does not appear naturally in members of the plant kingdom. So typically, the non-plant protein is of animal (in particular mammalian), viral (from virus that do not infect plants), bacterial, or fungal origin. In particular, non-plant polypeptides of interest in the present invention are mammalian polyepeptides/proteins, which are expression products of malignant cells, as well as polypeptides/proteins, which are expression products from pathogenic virus, bacteria, and fungi, in particular expression product from human pathogens. The non-plant polypeptide or protein can also be an artificial sequence that does not occur in nature.

The “origin” of a polypeptide or protein as defined herein is the one or more organisms or virus in which the polypeptide or protein-if not artificial-exists as a natural expression product or as a fragment thereof.

The term “subsequence” means any consecutive stretch of at least 3 amino acids or, when relevant, of at least 3 nucleotides, derived directly from a naturally occurring amino acid sequence or nucleic acid sequence, respectively. The term subsequence is used interchangeably with “fragment” in the context of nucleic acids and polypeptides.

The term “amino acid sequence” is the order in which amino acid residues, connected by peptide bonds, lie in the chain in peptides and proteins in the direction from the free N-terminus to the free C-terminus.

The term “adjuvant” or “immunological adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and immunological adjuvant induces an immune response against the immunogen which is stronger than that induced by the immunogen alone.

“Sequence identity” is in the context of the present invention determined by comparing 2 optimally aligned sequences of equal length (e.g. DNA, RNA or amino acid) according to the following formula: (N_{ref}−N_{dif}). 100/N_{ref}, wherein N_{ref} is the number of residues in one of the 2 sequences and N_{dif} is the number of residues which are non-identical in the two sequences when they are aligned over their entire lengths and in the same direction. So, two sequences 5′-ATTCGGAAC-3′ and 5′-ATACGGGAC-3′ will provide the sequence identity 77.8% (N_{ref}=9 and N_{dif}=2). It will be understood that such a sequence identity determination requires that the two aligned sequences are aligned so that there are no overhangs between the two sequences: each amino acid in each sequence will have to be matched with a counterpart in the other sequence.

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right-typically, the immunogenic carrier is capable of eliciting a strong T-helper lymphocyte response against the combined substance constituted by the immunogen and the immunogenic carrier, and this in turn provides for improved responses against the immunogen by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “linker” is an amino acid sequence, which is introduced between two other amino acid sequences in order to separate them spatially. A linker may be “rigid”, meaning that it does substantially not allow the two amino acid sequences that it connects to move freely relative to each other. Likewise, a “flexible” linker allows the two sequences connected via the linker to move substantially freely relative to each other. In fusion proteins, which are part of the present invention, both types of linkers are useful.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule presenting the peptide.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the are antigens bound to MHC molecules are being recognized by T-cell receptors) but which are not necessarily capable of inducing immunity—an antigen is, however, always capable of eliciting immunity, meaning that a host that has an established memory immunity against the antigen will mount a specific immune response against the antigen.

A “hapten” is a small molecule, which can neither induce nor elicit an immune response, but if conjugated to an immunogenic carrier, antibodies or TCRs that recognize the hapten can be induced upon confrontation of the immune system with the hapten carrier conjugate.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method disclosed herein substantially all will mount an immune response against the immunogen of the present invention.

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanised antibodies, altered antibodies, univalent antibodies, Fab proteins, and single domain antibodies.

“Specific binding” denotes binding between two substances which goes beyond binding of either substance to randomly chosen substances and also goes beyond simple association between substances that tend to aggregate because they share the same overall hydrophobicity or hydrophilicity. As such, specific binding usually involves a combination of electrostatic and other interactions between two conformationally complementary areas on the two substances, meaning that the substances can “recognize” each other in a complex mixture.

The term “vector” is used to refer to a carrier nucleic acid molecule into which a heterologous nucleic acid sequence can be inserted for introduction into a cell where it can be replicated and expressed. The term further denotes certain biological vehicles useful for the same purpose, e.g. viral vectors and phage-both these infectious agents are capable of introducing a heterologous nucleic acid sequence into cells.

The term “expression vector” refers to a vector containing a nucleic acid sequence coding for at least part of a gene product capable of being transcribed. In some cases, when the transcription product is an mRNA molecule, this is in turn translated into a protein, polypeptide, or peptide.

A “glycan” is a carbohydrate or chain of carbohydrate which is linked to biomolecules (such as to lipids or proteins).

“High-mannose” in the present context denotes a Man5-Man9 glycosylation pattern.

### Glycobiology

Glycobiology is described as the biology, biosynthesis, structure and evolution of saccharides that are widely distributed in nature and of the proteins that recognize them. Saccharides are also called carbohydrates or sugar chains. All cells and numerous macromolecules in nature carry an array of covalently attached sugars or glycosidically linked sugar chains, which are referred to as “glycans”.

### Common Monosaccharide Units of Glycoconjugates

Monosaccharides have been found in hundreds of different versions in nature. However, in common glycans the monosaccharide variation is limited to the saccharides mentioned in the following table:

| Saccharides | Explanation | Example |
|---|---|---|
| Pentoses | Five-carbon neutral sugars | D-xylose |
| Hexoses | Six-carbon neutral sugars | D-glucose |
| Hexosamines | Hexoses with an amino group | N-acetyl-D- |
| | at the 2-position, which can | glucosamine |
| | be either free or N-acetylated | |
| 6-Deoxyhexoses | | L-fucose |
| Uranic acids | Hexoses with a carboxylate group | D-glucuronic |
| | at the 6-position | acid |
| Nonulosonic | Family of nine-carbon acidic | Sialic acids |
| acids | sugars | |

### Glycosidic Linkages

Monosaccharides are linked together via glycosidic bonds. The anomeric carbon of each saccharide is a stereocenter, which means that each glycosidic linkage can be constructed as either an α- or a β-linkage. Depending on which carbon atom in the sugar structure the binding occurs to, the name can be for example either Mana1,6 or Mana1,3, occurring on the 6^{th} or the 3^{rd} carbon atom respectively.

### Glycan-Processing Enzymes

Generally, there are two groups of glycan-modifying enzymes the transferases and the glycosidases. The glycosyltransferases assemble branched and linear glycan chains and link monosaccharide moieties together. Glycosidases have the opposite effect; they degrade glycan structures, either for turnover of used glycans or as intermediates used as substrates in biosynthesis of glycans. The glycosyltransferases are generally specific in both donor and acceptor substrates. For example, the α2,3-sialyltransferase acts on β-linked galactose and the β1,4-galactosyltransferase acts on β-linked N-acetylglucosamine (GlcNAc).

### Types of Glycosylation

Glycosylation is a broad term and covers several different types of oligosaccharides and linkages. Glycosylation is found in all domains of life, and they vary greatly in structure across these domains. Bacteria have glycans on their surface. The most recognized is lipopolysaccharide (LPS) also known as “endotoxin” that is found on the surface of the outer membrane of Gram-negative bacteria. Gram-positive bacteria have capsular polysaccharide among other glycans on their cell wall. Archaea also carry glycans on the surface layer of their cell wall and they can even carry out N-glycosylation of proteins. The glycosylation in eukaryotic cells is more extensively studied and the major glycan-categories in mammalian cells are Glycosphingolipids, Proteoglycans, N-linked glycans, and O-linked glycans. See FIG. 2.

### Glycolipids

Glycolipids are lipids with a glycan attached by a glycosidic bond. They are generally found on the extracellular surface of eukaryotic cell membranes. Here, they extend from the phospholipid bilayer and out into the extracellular space. Glycolipids maintain stability of the membrane and aid in cell-to-cell interactions. Furthermore, glycolipids can act as receptor for viruses and other pathogens to enter cells. Glycerolipids and sphingolipids are the two most common types of glycolipids.

### Proteoglycans

Proteoglycans are heavily glycosylated proteins found on the extracellular side of animal cell membranes. Proteoglycans consists of a core protein and one or more covalently bound linear glycosaminoglycan chains. They fill out the space between cells in a multicellular organism and play significant roles in matrix assembly, modulation of cellular signals, and serve as a reservoir of biologically active small proteins such as growth factors.

### O-Linked Glycosylation

The broad description of O-linked glycosylation is the attachment of a saccharide to an oxygen atom of an amino acid residue in a protein, most often serine and threonine. O-linked glycans are constructed by the addition of O—N-acetylgalactosamine, O-fucose, O-glucose, O—N-acetylglucoasmine or O-mannose. Hyper-O-glycosylation can result in the formation of mucin-type molecules that coat mucosal surfaces. Initially attached N-acetylglucosamine (mucin-type O-glycosylation) or mannose (O-mannosylation) are often prolonged (linear or branched) by 5-10 different monosaccharides, such as galactose, N-acetylglucosamine, N-galactosamine, sialic acid and xylose. For N-acetylglucosamine based mucin-type O-glycans, eight different cores structures are known today.

### N-Linked Glycosylation

It is known that the structure, number, and location of N-glycans can affect the biologic activity, protein stability, clearance rate and immunogenicity of biotherapeutic proteins. N-linked glycans are most often found on cell surfaces and on secreted proteins. The N-glycosylation on proteins can occur on the amino acid sequence of a protein where an Asn precedes any amino acid but Pro, which is in turn followed by Ser or Thr. The common N-glycan “core” structure shared between all eukaryotic cells is Mana1-3(Mana1-6)Manβ1-4GlcNAcβ1-4GlcNAcβ1-Asn-X-Ser/Thr. Different organisms build differently onto this core structure and the glycans are categorized into 1) “oligomannoses”, where only mannose residues extend the antennas; 2) “complex”, where initially GlcNAcs extend the core; 3) “hybrid”, where Man extends the Mana1-6 arm of the core and GlcNAc extends the Mana1-3 arm.

N-Linked Glycan Nomenclature in this Application

Describing N-glycans in writing can cause some confusion. The level of necessary detail and information can vary between situations. Sometimes it is necessary to know each branching and linkage type, and sometimes it is only necessary to communicate whether a structure has e.g., four or five mannoses. There has been no consensus up until recently, and authors have either invented their own nomenclature or modified an existing one. To avoid confusion, the present application will use the “Oxford notation”, which is based on building up N-glycan structures. Therefore, it can be used to denote very complex glycans. In brief, the notation is as follows:

All N-glycans have two core GlcNAcs; F at the start of the abbreviation indicates a core fucose; Mx, number (x) of mannose on core GlcNAcs; Ax, number of antenna (GlcNAc) on trimannosyl core; “A2”, biantennary with both GlcNAcs as alpha1-2 linked; Gx, number (x) of linked galactose on antenna; [3]G1 and [6]G1indicates that the galactose is on the antenna of the α1-3 or α1-6 mannose; Sx, number (x) of sialicacids linked to galactose.

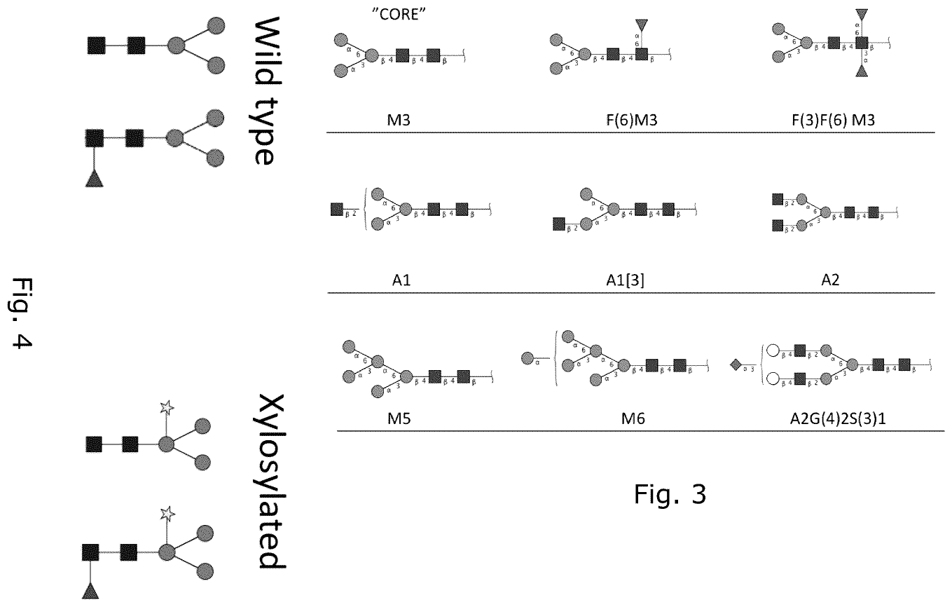

Examples of the most commonly occurring N-linked glycans in this application are provided in FIG. 3.

The Oxford nomenclature is relatively intuitive. The “core” consists of two GlcNAc residues and three mannose residues. The first GlcNAc is linked to the Asn amino acid by a β-linkage. The next GlcNAc is linked by β1,4-linkage to a mannose, followed by a β1,4-linked mannose. From here, the glycan structure branches and the two remaining mannoses are attached by either an α1,3-linkage or an α1,6-linkage. This core is ubiquitous in N-glycans and is named “M3”. If it has a core fucose it is called “FM3”. If the position is known, then it is written in parenthesis e.g. “F(6)M3” in the case where the fucose is α1,6-linked, “F(3)M3” in the case where the core fucose is α1,3-linked, or e.g. “F(3) F(6)M3” in the case where the core has both a α1,3-linked and a α1,6-linked fucose. Once the sugars are added to this core, the name depends on these. The core with one GlcNAc is called “A1”. If the position is known, then it is added in square brackets, e.g. “A1 [3]” if the GlcNAc is on the α1,3-linked mannose branch. If the glycans fall into the “high-mannose” category, then some structures are “fixed” both structurally and nomenclature-wise, like “M5” and for others like “M6” the position of the added mannose residue can vary. There are names for complex tri- and tetra-antennary structures, where every linkage and position is defined. An example of a more complex structure is the “A2G(4)2S(3)1”, where the “A2” describes the two β1,2-linked GlcNAcs, the “G(4)2” describes the 2 galactoses that are both β1,4-linked (and not e.g. α1,3-linked), and the “S(3)1” describes one sialic acid linked by a α2,3-link. If the position was known, then it would be indicated with a “[3]” or “[6]” referring to the α1,3- or α1,6-linked mannose branch.

### N-Glycan Synthesis

The category of N-glycan that is found on a protein depends on the organism, from which it originates. All N-glycans, whether in yeast, insect cells or mammalian cells, start out as the same structure in the endoplasmic reticulum lumen. N-glycan synthesis occurs in two steps. 1) Synthesis and transfer of a dolichol-linked precursor and 2) processing steps of the Glc3Man9GlcNAc2Asn glycan.

### Synthesis and Transfer of the Dolichol-Linked Precursor

The first part of N-glycosylation of a protein is the construction of the Dolichol-precursor and the attachment of this to an asparagine residue of the protein. This is described in detail below.

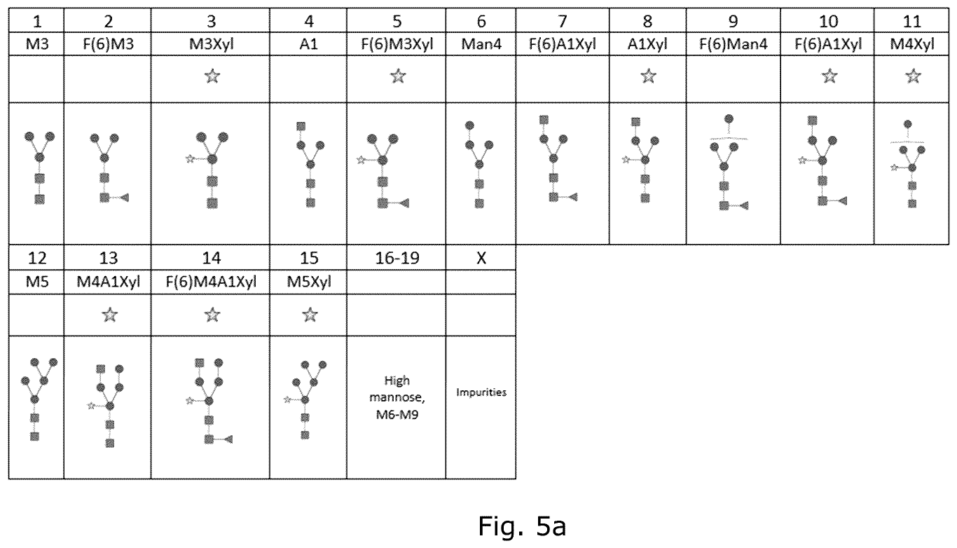

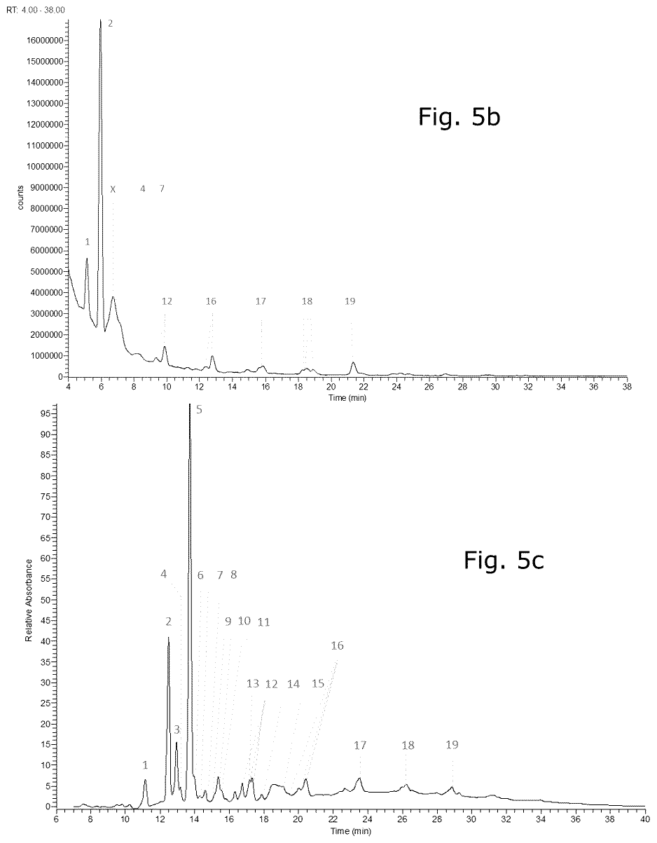

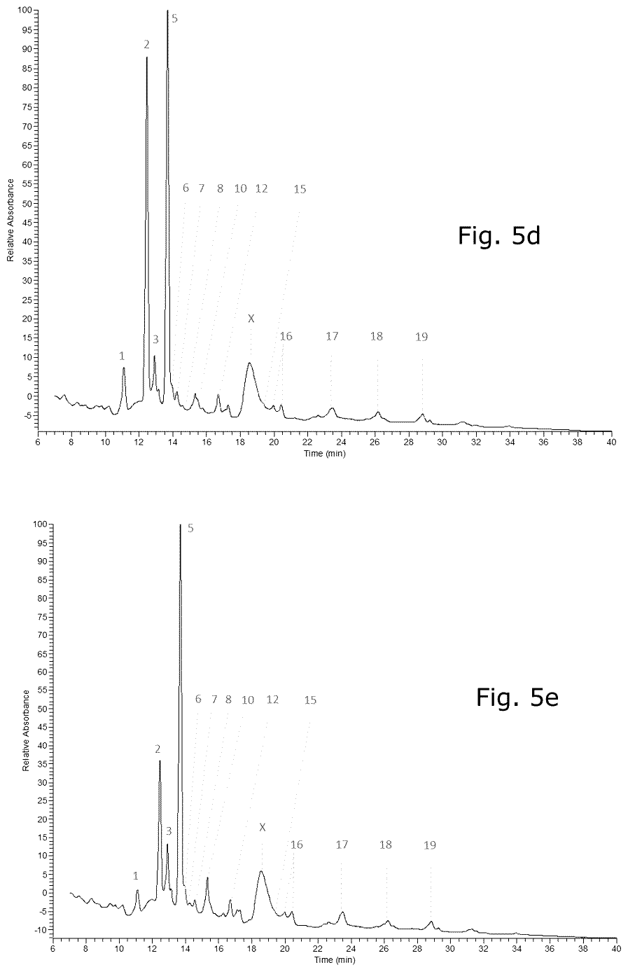

Dolichol phosphate is located on the cytoplasmic side of the membrane of the endoplasmic reticulum (ER). Dolichol phosphate receives GlcNAc-1-P from UDP-GlcNAc to make Dol-P-P-GlcNAc, which is then extended to Dol-P-P-M5. At this point an enzyme called “flippase” flips the structure to the inside of the ER lumen and four Man residues from Dol-P-Man and three Glc residues from Dol-P-Glc are added. This oligosaccharide is transferred to the Asn residue of a protein within the sequence pattern N-X-S/T by an oligosaccharyltransferase that covalently binds the glycan to the protein. See FIG. 5.

### Processing Steps of the M9Glc3 Glycan

Once the protein is equipped with the M9Glc3 glycan at the N-glycan sites, the processing starts. Common for most eukaryotic organisms is that the protein is folded and transported to the Golgi apparatus for further N-linked glycan processing. Depending on the organism there are different pathways and enzymes responsible for the final N-linked glycan structure. The initial de-glucosylation is carried out by α-glucosidase I, which removes the first α1,2-linked glucose residue. The next glucose is α1,3-linked and removed by α-glucosidase II.

After removal of these two glucose residues the N-linked glycan processing pathway intersects with the protein quality control pathway to ensure proper folding of the newly synthesized proteins carrying M9Glc1. The quality control pathway is mainly mediated by the ER chaperones calnexin and calreticulin. These two chaperones require the presence of the α1,3-linked glucose residue to bind to the protein. As soon the last glucose residue is removed, the chaperones terminate their folding process. This step leaves either a correctly or incorrectly folded protein with M9. The incorrectly folded proteins are re-glucosylated by a glucosyltransferase resulting in a monoglucosylated form that can again bind to chaperones. If proper folding ultimately fails the protein is degraded in a separate ER compartment, the ER-associated degradation pathway (ERAD). Correctly folded glycoproteins are finally processed by a class I α-mannosidase which removes the α1,2-mannose on the B-branch. Now, the glycoprotein, which carries M8, is transported to the Golgi where the remaining glyco-processing takes place. The proteins are delivered to the cis-side of the Golgi and are modified as they move through the medial to the trans Golgi cisternae. The pathway from now on depends on whether the N-linked glycosylation is taking place in yeast, plants, insects or mammals.

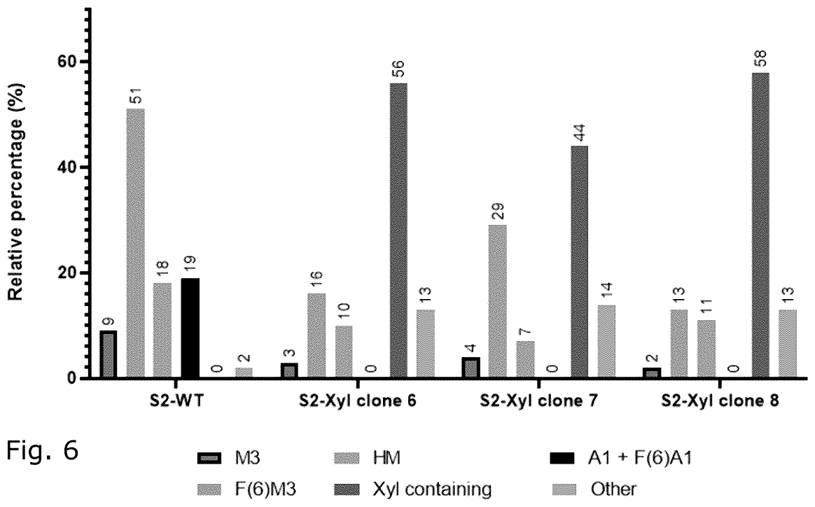

This is where N-linked glycans are split up in “oligomannose”, “complex”, or “hybrid” as described in FIG. 3. Biosynthesis of complex and hybrid N-linked glycans is initiated in the medial-Golgi N-acetylglucosaminyltransferase I (GlcNACT I), which adds GlcNAc to the second carbon atom of the α1,3-Man in the core or M5. Next, the two mannoses on the 6-branch are cleaved off by α-mannosidase II to yield A1. α-mannosidase II can only act after the action of GlcNACT I, as it is substrate specific for A1M5. The resulting A1 is the point where invertebrates and plants start separating from mammals. The genome of plants and invertebrates, including insects, encodes a hexoaminidase by the fused lobes gene (fdl) that removes the terminal GlcNAc residue and forms M3. In contrast, the mammalian cells encode N-acetylglucosaminyltransferase II (GlcNACT II) that adds a GlcNAc on the 6-branch and thus forms A2. This structure is then further extended to contain galactose and sialic acids. See FIG. 6. For some mammalian glycoproteins tri- or tetra-antennary structures are also found. The major core modification in both mammalian, invertebrate and plant glycans is the attachment of core fucose. In plants and some insect cells, core fucose is often added by a α1,3-linkage and in other insect cells and mammalian cells it is added by a α1,6-linkage. Similar to α-mannosidase II, α1,6-fucosyltransferase also requires the preceding action of GlcNAcT I to function. In plants, the addition of β1,2-xylose to the β-Man of the core is also common.

Plant glycosylation is reviewed by Strasser R. 2016 (Glycobiology 26, 926-939). The pathway progresses like mammalian glycosylation up until the medial-golgi cisternae resulting in A1M5. Here similar to most other eucaryotic organisms, the A1M5 precursor is trimmed to A1. In plants the processing can continue with xyIT using either A1M5, followed by trimming of mannoses, or A1 as substrate resulting in A1 xylosylated at the α-Man of the core. As in mammals GlcNAcTII then adds β1,2-GlcNAc to the 6-branch resulting in A2 with xylose β1,2-linked to α-Man (Kajiura et al. 2012, J. Biosci. Bioeng. 113, 48-54). Unlike mammals and insects however, the plant glycans are not further extended and do not branch beyond bi-antennary structures. Further in the trans-Golgi cisternae the glycans can be α1,3-fucosylated by fut11 or fut12. There are some unknown factors for plant glycosylation. For example, post-Golgi glycomodification in the vacuole has been proposed and the function of glycosylation in plants grown under normal conditions is not obvious. Glycosyltransferases are conserved between organisms spanning mosses and higher plants indicating that evolutionary constraints keep them from being deleted. But in studies where glycosylatransferases, including α1,3-fucosyltransferases and β1,2-xylosyltransferases, were deleted the phenotype was unchanged or only detected under stress conditions (Koiwa et al. 2003, Plant Cell 15, 2273-2284, Strasser R2016).

The following abbreviations are used throughout the present application:
α-gal: galactose-α1,3-galactose
BEVS: Baculovirus Expression Vector System
BHK21: Baby Hamster Kidney Cell
Cas9: CRISPR Associated protein 9
CE: Capillary Electrophoresis
CHO: Chinese Hamster Ovary Cells
CLR: C-type Lectin Receptor
ConA: Concanavalin A
CRISPR: Clustered Regularly Interspaced Short Palindromic Repeats
DC: Dendritic Cell
DC-SIGN: Dendritic Cell Specific Intercellular adhesion molecule-3-Grabbing Non-integrin
Ebola GP1: Ebola Glycoprotein 1
ESI: Electron spray ionization
FAB: Fast atom bombardment
Fab: Antigen binding fragment (of antibodies)
Fc: Constant fragment (of antibodies)
fdl: fused lobes gene
FucT6: α1,6-fucosylatransferase gene
fut11: α1,3-fucosylatransferase gene
GalNAc: N-acetylgalactosamine
GlcNAc: N-acetylglucosamine
GlcNACT I: N-acetylglucosaminyl transferase I gene
GlcNACT II: N-acetylglucosaminyl transferase II gene
HA: hemagglutinin
hEPO: human erythropoietin
HER2: Human epidermal growth factor receptor 2
HILIC: Hydrophilic interaction chromatography
HM: High-mannose
ID1-ID2a: Interdomain 1-Interdomain 2a (of VAR2CSA).
Indel: Insert/deletion
LC-MS: Liquid Chromatography Mass Spectrometry
LCA: Lens culinaris agglutinin
LPS: Lipopolysaccharide
M3 (or “Man₃”): refers to the “core” structure of glycans
mAb: monoclonal antibody
MALDI-TOF: Matrix-assisted laser desorption/ionization Time of Flight
MGAT4: N-acetylglucosaminyl transferase IV gene
MGAT5: N-acetylglucosaminyl transferase V gene
MHC: Major histocompatibilty complex
mo-DC: monocyte derived dendritic cell
MPLA: Monophosphoryl lipid A
MR: Mannose receptor
MS: Mass spectrometry
Neu5Gc: N-glycolylneuraminic acid
NHEJ: Non-homologous end joining
PAM: Protospacer Adjacent Motifs
PM: Placental Malaria
PRR: Pattern recognition receptors
PTM: Post-translational modification
QIT: Quadrupole Ion Trap
S2: Drosophila melanogaster Schneider 2 cells
S3: Drosophila melanogaster Schneider 3 cells
SDS-PAGE: sodium dodecyl sulfate polyacrylamide gel electrophoresis
SPR: Surface Plasmon Resonance
TLR: Toll-like Receptor
VAR2CSA: Receptor of malaria infected erythrocytes, which bind to placenta cells
VLP: Virus-like particle
WT: Wild-type

### 1^{st} Aspect of the Invention and Embodiments Thereof

The 1^{st} aspect of the invention relates as indicated above to a non-plant polypeptide or protein comprising xylosylated N-linked glycans that comprise β1,2-xylose, wherein at least 25% of the individual protein species comprise xylosylated N-glycans.

In some embodiments, the non-plant polypeptide or protein is selected from the group consisting of VAR2CA, HER2 and hEPO; a viral protein or polypeptide from HIV, Ebola virus, Zika virus, Chikungunya virus, Dengue virus, Hepatitis A virus, influenza virus, Polio virus, Rabies virus, Measles virus, mumps virus, rubella virus, Rotavirus virus, Smallpox virus, Chickenpox virus, Hepatitis B virus, human papillomavirus, varicella zoster virus, Yellow fever virus, SARS-CoV-1, cytomegalovirus (CMV), and SARS-CoV-2; and a bacterial protein or polypeptide from Clostridium tetanii, Corynebacterium diphtheria, Haemophilus influenzae, Bordetella pertussis, Streptococcus pneumoniae, and Neisseria meningitides. As mentioned herein, these proteins and polypeptides are all associated with diseases and the provision of the proteins and polypeptides of the invention hence also provides the possibility of treating or reducing the risk of developing the diseases associated with the wild-type of the proteins and polypeptides.

The non-plant polypeptide or protein in some embodiments comprises F(6)M3, F(6)A1, F(6)A2 and/or F(3) F(6)M3 glycan structures and/or comprises increased Man-5-Man9 structures compared to the its non-plant wild-type protein counterpart (if such a wild-type protein exists).

As indicated above, the non-plant polypeptide or protein of the 1^{st} aspect and the embodiments discussed above is of mammalian, viral, fungal or bacterial origin, but can also be of crustacean, insect, arachnoid, helminthic, or protozoan origin.

The glycosylation obtained according to the invention is such that a non-plant polypeptide or protein of the first aspect has at least 25% of individual protein/peptide species comprise xylosylated N-glycans, such as at least 35%, at least 40%, at least 45%, at least 50%, at least 55%, at least 60%, at least 65%, at least 70%, 75%, and at least 90%. This is to mean that the glycosylation pattern naturally varies from molecule to molecule having the same amino acid sequence, but that the preferred polypeptide or protein of the present invention is one where the β1,2-xylose (and preferably high mannose glycans and/or fucosylated glycans, see below) appear in a large proportion of the molecules having the same amino acid sequences.

As detailed in the example, a non-plant polypeptide or protein of the first aspect of the invention has been produced in S2 cells. Hence, in one preferred embodiment, the non-plant polypeptide or protein is obtainable or obtained by a method comprising expressing polynucleotide(s) encoding the polypeptide or protein in an S2 cell transformed so as to produce an active β1,2-xylosyltransferase-such an S2 cell can e.g. be the S2 cell line S2-Xyl disclosed herein, the S2 cell line M3Xyl or the S2 cell line F(6)M3Xyl. In addition, and as outlined in Example 2, the cell line can further be genetically altered to produce an active UDP-xylose synthase or exhibit increased expression of the UDP-xylose synthase gene. The latter may be accomplished by introducing an active (optionally heterologous) gene encoding the UDP-xylose synthase or by modifying expression control of a UDP-xylose synthase gene already present in the genome of the cell line. For instance, the expression control region for an existing UDP-xylose synthase gene can be genetically modified by e.g. introducing a stronger promoter/enhancer region, or the transcription product can be rendered more stable to exhibit a longer half-life in vivo and thereby effect increases in the amount of translation product. Tools, such as CRISPR-Cas9 technology, for site-specific recombinant modification of cells are well known to the person skill in the art.

Apart from comprising xylosylated N-linked glycans that comprise β1,2-xylose, the non-plant polypeptide or protein of the first aspect can contain further glycosylation modifications. For instance, the non-plant polypeptide or protein may also comprise α1,3-linked fucose. In that case, the protein or polypeptide may also contain F(3)M3 glycan structures.

For details about non-plant polypeptide or protein may also comprise α1,3-linked fucose as well as increased Man-5-Man9 structures, reference is made to the disclosure in WO 2020/144358, the disclosure of which is hereby incorporated by reference herein.

The present non-plant polypeptide or protein of the first aspect of the invention finds i.e. use as an immunization antigen in mammals, cf. the example. Therefore, in some embodiments the non-plant polypeptide or protein of the first aspect of the invention further comprises or is coupled via a non-peptide bond to a heterologous moiety, such as a purification tag, an immunogenic carrier molecule or T-helper lymphocyte epitope, a solubility-modifying group, a protraction group, a targeting moiety, a virus-like particle, and an immune modulating moiety, said heterologous moiety optionally being fused to the polypeptide or protein via a peptide linker. Such a heterologous moiety imparts a further functionality to the polypeptide or protein: increased immunogenicity, ease of purification, increased biological half-life etc. In some cases it is practical to use a “linker” (rigid or flexible as the need may dictate), i.e. a relatively short stretch of amino acids instead of coupling the moiety directly to the protein/polypeptide—this can e.g. be the case in order to avoid steric interactions between the moiety and the protein/polypeptide.

### Expression Systems

Eukaryote-based systems can be employed for use with the present invention to produce N-glycosylated polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ Baculovirus expression system from CLONTECH®

In addition to the disclosed expression systems disclosed herein, other examples of expression systems include STRATAGENER's COMPLETE CONTROL™ Inducible Mammalian Expression System, which involves a synthetic ecdysone-inducible receptor. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### 2^{nd} Aspect of the Invention and Embodiments Thereof

Immunogenic compositions, in particular vaccines, according to the invention may be prophylactic (i.e. suited to prevent infection) or therapeutic (i.e. to treat disease after infection) or they may be useful for induction of antibody production in animals used for that purpose.

The immunogenic composition of the invention comprises a non-plant polypeptide or protein according to the first aspect of the invention (or a polypeptide or protein produced according to the method of the 5^{th} aspect of the invention) in admixture with at least one immunological adjuvant and optionally a pharmaceutically acceptable carrier and/or diluent and/or excipient; see below for the detailed discussion of immunological adjuvants and other substances in the composition.

Typically, an immunogenic composition of the invention is in the form of a liquid formulation (suitable for injection or ingestion) such as a solution, a suspension, an emulsion, or a suspoemulsion, or in the form of a solid or semisolid formulation, such as a powder, tablet, suppository, pill, gel, cream, or ointment. Conveniently, an immunogenic composition of the invention is contained a unit dose form, such as in freeze-dried form.

Vaccines disclosed herein typically comprise immunising N-glycosylated polypeptide(s), protein(s) or peptide(s), usually in combination with “pharmaceutically acceptable carriers”, which include any carrier that does not itself induce the production of antibodies harmful to the individual receiving the composition or targeting the protein/pathogen. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles.

Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, etc. pathogen, cf. the description of immunogenic carriers supra.

The pharmaceutical compositions disclosed herein thus typically contain an immunological adjuvant, which is commonly an aluminium based adjuvant (including an aluminium salt), an oil-in-water emulsion, a saponin, complete and incomplete Freund's adjuvant, and a cytokine; or one of the other adjuvants described in the following:

Preferred adjuvants to enhance effectiveness of the composition include, but are not limited to:
(1) aluminium salts (alum), such as aluminium hydroxide, aluminium phosphate, aluminium sulfate, etc;
(2) oil-in-water emulsion formulations (with or without other specific immunostimulating agents such as muramyl peptides (see below) or bacterial cell wall components), such as for example (a) MF59 (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, MA), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, (c) AddaVax™ (a squalene-based oil-in-water nano-emulsion similar to MF59, and (d) Ribi adjuvant system (RAS), (Ribi Immunochem, Hamilton, MT) containing 2% Squalene, 0.2% Tween 80, and one or more bacterial cell wall components from the group consisting of monophosphoryl lipid A (MPL), trehalose dimycolate (TDM), and cell wall skeleton (CWS), preferably MPL+CWS (Detox™);
(3) saponin adjuvants such as Stimulon™ (Cambridge Bioscience, Worcester, MA) may be used or particles generated therefrom such as ISCOMs (immunostimulating complexes);
(4) Complete Freund's Adjuvant (CFA) and Incomplete Freund's Adjuvant (IFA);
(5) cytokines, such as interleukins (eg. IL-1, IL-2, IL-4, IL-5, IL-6, IL-7, IL-12, etc.), interferons (e.g. gamma interferon), macrophage colony stimulating factor (M-CSF), tumor necrosis factor (TNF), etc.; and
(6) other substances that act as immunostimulating agents to enhance the effectiveness of the composition. Alum, MF59™, and Addavax™ adjuvants are preferred, but MPLS/LPS and TLR4 agonists are other possibilities.

Muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2″-2′-dipalmitoyl-sn-glycero-3-hydroxyphosphoryloxy)-ethylamine (MTP-PE), etc.

The immunogenic compositions (e.g. the immunising antigen or immunogen or polypeptide or protein or nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic or immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individuals to be treated (eg. nonhuman primate, primate, etc.), the capacity of the individual's immune system to synthesize antibodies or generally mount an immune response, the degree of protection desired, the formulation of the vaccine, the treating doctor's assessment of the medical situation, and other relevant factors. It is expected that the amount will fall in a relatively broad range that can be determined through routine trials. However, for the purposes of protein vaccination, the amount administered per immunization is typically in the range between 0.5 μg and 500 mg (however, often not higher than 5,000 μg), and very often in the range between 10 and 200 μg.

The immunogenic compositions are conventionally administered parenterally, eg, by injection, either subcutaneously, intramuscularly, or transdermally/transcutaneously (e.g. as in WO 98/20734). Additional formulations suitable for other modes of administration include oral, pulmonary and nasal formulations, suppositories, and transdermal applications.

Dosage treatment may be a single dose schedule or a multiple dose schedule. The vaccine may be administered in conjunction with other immunoregulatory agents.

### 3^{rd} Aspect of the Invention and Embodiments Thereof

As mentioned above, the proteins and polypeptides of the first aspect of the invention enables the method of the 3^{rd} aspect for inducing/enhancing a specific immune response in an animal, such as a human being, the method comprising at least one immunization of the animal with an effective amount of the protein or polypeptide of the first aspect or with the composition of the 2^{nd} aspect.

Irrespective of the precise protein or polypeptide, it is preferred to administer the active principle in both a priming immunization and at least one subsequent booster immunization. Alternatively, the somewhat more recent approach of utilising cluster immunizations (i.e. an immunization scheme where repeated dosages of the immunogen(s) are administered at short intervals in the beginning of the immunization regimen before a memory immune response has been established; this is then followed by delay immunizations that resemble the traditional booster immunization used in a prime-boost immunization regimen).

The disease targeted by immunization naturally depends on the origin of the immunogen. For instance, in some embodiments of the present invention the at least one immunization reduces risk in the vaccinated animal of attracting a disease caused by an infectious organism or where the immunization modulates an existing immune response against the protein or polypeptide—the latter is e.g. relevant when treating allergies by specific immune therapy, in which case the undesired Th2 dependent IgE immune response is modulated into an non-harmful Th1 dependent IgG immune response.

In other embodiments of the 3^{rd} aspect, the at least one immunization treats or ameliorates or reduces risk of disease caused by an autologous protein or by a cell producing said autologous protein. This is e.g. relevant in cancer immune therapy, where cancer-associated or cancer-specific antigens can be targeted, but also when immunizing actively against autologous proteins that in their own right contribute to progression of disease.

Immunization routes are typically selected from parenteral routes such as the subcutaneous, intradermal, subdermal, intraperitoneal, intrathecal, and intramuscular routes, or via the oral or oral mucosal routes. See below for details.

To conclude the method of the 3^{rd} aspect is normally selected from
a) a method for disease prophylaxis;
b) a method for treatment or amelioration of disease; and
c) a method for antibody production.

Related to the third aspect is also the protein or polypeptide of the 1^{st} aspect of the invention or the composition of the second aspect for use in a prophylactic or therapeutic method of the 3^{rd} aspect. Likewise, a related aspect is the use of a protein or polypeptide of the first aspect for the preparation of a pharmaceutical composition (of the second aspect) for use in a therapeutic or prophylactic method of the 3^{rd} aspect.

The 3^{rd} aspect of the invention generally relates to induction of immunity and as such also entails method that relate to treatment, prophylaxis and amelioration of disease as well as to methods the aim at producing antibodies in a host animal.

When immunization methods entail that a polypeptide disclosed herein or a composition comprising such a polypeptide is administered the animal (e.g. the human) typically receives between 0.5 and 5,000 μg of the polypeptide disclosed herein per administration, cf. above.

In preferred embodiments of this aspect, the immunization scheme includes that the animal (e.g. the human) receives a priming administration and one or more booster administrations.

Preferred embodiments of this aspect disclosed herein comprise that the administration is for the purpose of inducing protective of therapeutic immunity against an infectious agent. Alternatively, the administration is aimed at preventing or treating diseases caused by autologous proteins or cells; such diseases include (malignant) neoplastic diseases but also diseases where autologous proteins induce undesirable side effects.

The compositions disclosed herein can induce humoral immunity, so the administration is in some embodiments for the purpose of inducing antibodies specific for the antigen, cell or organism from which the glycosylated polypeptide or protein is derived, and wherein said antibodies or B-lymphocytes producing said antibodies are subsequently recovered from the animal to be used in their own right as pharmaceutical, diagnostic or laboratory agents.

Pharmaceutical compositions can as mentioned above comprise polypeptides/proteins disclosed herein. The pharmaceutical compositions will comprise a therapeutically effective amount thereof.

The term “therapeutically effective amount” or “prophylactically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. Reference is however made to the ranges for dosages of immunologically effective amounts of polypeptides, cf. above.

However, the effective amount for a given situation can be determined by routine experimentation and is within the judgement of the clinician.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N. J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Diseases or infections targeted are for instance SARS-CoV-2 infections (and thus the disease COVID-19), influenza, and CMV infections. In general, each of the microorganisms mentioned for the proteins of the first aspect of the invention are targets for immunization with the respective proteins and composition of the first aspect of the invention.

### 4^{th} Aspect of the Invention and Embodiments Thereof

Genetically modified cells disclosed herein are useful as organisms for producing proteins and polypeptides, typically proteins and polypeptides of the first aspect of the invention.

A genetically modified non-plant eukaryotic cell, such as an insect or mammalian cell or a fungal cell such as a yeast, of the 4^{th} aspect is one that which comprises at least one heterologous polynucleotide sequence encoding and expressing β1,2-xylosyltransferase, wherein the cell is capable of producing N-glycosylated protein carrying β1,2-xylose groups, and where the heterologous polynucleotide sequence preferably is the XylT gene from Arabidobsis thaliana, or an equivalent polynucleotide encoding a plant β1,2-xylosyltransferase.

As demonstrated in Example 2, embodiments of the 4^{th} aspect entails that the genetically modified non-plant eukaryotic cell comprises at least one (optionally heterologous) polynucleotide sequence encoding and expressing a UDP-xylose synthase or comprises at least one additional (optionally heterologous) UDP-xylose synthase encoding sequence so as to exhibit increased UDP-xylose synthase activity. For instance, the encoded UDP-xylose synthase can be the one having SEQ ID NO: 6; one example of a coding sequence is SEQ ID NO: 5.

In some embodiments, the genetically modified non-plant eukaryotic cell of the 4^{th} aspect further comprises at least one heterologous polynucleotide sequence encoding and expressing a heterologous α1,3-fucosyltransferase, and wherein expression of the α-Man-Ia gene optionally has been reduced or abolished or wherein expression of genes encoding enzymes extending glycans beyond Man3, such as beyond Man5 optionally has been increased, wherein the cell is capable of producing N-glycosylated protein carrying α1,3-fucosyl groups, and which preferably exhibits reduced or abolished function of at least one α1,6-fucosyltransferase encoding gene, and where the heterologous polynucleotide sequence preferably is the fuc11 gene from Arabidobsis thaliana, or an equivalent polynucleotide encoding a plant α1,3-fucosyltransferase. For details relating to these modifications, cf. WO 2020/144358.

While the most preferred non-plant eukaryotic cell of the 4^{th} aspect is an insect cell (such as a Drosophila S cell), the specific modifications introduced are also relevant in a range of other non-plant cell types, such as mammalian cells and fungal cells such as a yeast of filamentous fungal cells.

For production of protein and polypeptides, it is necessary that the genetically modified cell further expresses a (heterologous) gene encoding a polypeptide or protein, preferably one of the non-plant polypeptides or proteins of the first aspect of the invention

As mentioned above, the preferred genetically modified cell of the 4^{th} aspect is an insect cell, preferably a Drosophila melanogaster cell, such as an S2 or S3, or an insect cell such as Sf9, SF21, High5, and C6-36. However, in the event the cell is mammalian, it can be a CHO or HEK cell.

Useful cells are in general discussed in the following.

Eukaryotic cells can be in the form of yeasts (such as Saccharomyces cerevisiae) and protozoans. Alternatively, the transformed eukaryotic cells are derived from a multicellular organism such as a fungus, an insect cell, or a mammalian cell.

For production purposes, it is advantageous that the genetically modified cell disclosed herein is stably transformed by having the nucleic acids disclosed above stably integrated into its genome, and in certain embodiments it is also preferred that the genetically modified cell secretes or carries on its surface the glycosylated polypeptide disclosed herein, since this facilitates recovery of the polypeptides produced.

As noted above, stably genetically modified cells are preferred—these i.a. allows that cell lines comprised of genetically modified cells as defined herein may be established—such cell lines are particularly preferred aspects of the invention.

It is noted that the genetically modified cell of the 4^{th} aspect can be established as a cell line comprising the genetically modified cell; one example of such a cell line is a clonal cell line.

Further details on cells and cell lines are presented in the following:

Techniques for recombinant gene production, introduction into a cell, and recombinant gene expression are well known in the art. Examples of such techniques are provided in references such as Ausubel, Current Protocols in Molecular Biology, John Wiley, 1987-2002, and Greene and Sambrook: “Molecular Cloning: A Laboratory Manual (Fourth Edition)”, Cold Spring Harbor Laboratory Press (ISBN-10:9781936113422).

As used herein, the terms “cell”, “cell line”, and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A genetically modified cell includes the primary subject cell and its progeny.

Host cells are in the present application derived from non-plant eukaryotes, including yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials or from other depository institutions such as Deutsche Sammlung vor Microorganismen und Zellkulturen (DSM). An appropriate host can be determined by one of skill in the art based on the vector backbone and the desired result. A plasmid or cosmid, for example, can be introduced into a prokaryote host cell for replication of many vectors or expression of encoded proteins.

Examples of eukaryotic host cells for replication and/or expression of a vector include HeLa, NIH3T3, Jurkat, HEK293, Cos, CHO, Saos, and PC12. Many host cells from various cell types and organisms are available and would be known to one of skill in the art. Similarly, a viral vector may be used in conjunction with a host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above-described host cells to maintain them and to permit replication of a vector. Also understood and known are techniques and conditions that would allow large-scale production of vectors, as well as production of the nucleic acids encoded by vectors and their cognate polypeptides, proteins, or peptides.

### 5^{th} Aspect of the Invention and Embodiments Thereof

In the method for producing an N-glycosylated polypeptide or protein carrying β1,2-xylose, described under the Summary of the Invention heading above, any of the proteins disclosed in the first aspect may be prepared, and any of the cell lines of the 4^{th} aspect may function as host cells. In general, the method of the 5^{th} aspect relies on methods generally known in the art for cell culture, recombinant expression, and protein purification.

### EXAMPLE 1

Establishment of S2 Cell Lines Producing Proteins with Xylosylated N-Glycans; Immunization of Mice with Recombinant Protein Produced by the Cell Lines

### Plasmid Construction

The β1,2-xylosyltransferase gene from Arabidopsis thaliana (Accession Number NP_568825, SEQ ID NO: 1) was cloned into pExpres2-2 plasmid with G418 resistance gene (ExpreS2ion Biotechnologies, Denmark).

The construct was named ‘XylT, pExpreS2-2’. The gene for human erythropoietin (Accession Number AGW15567) flanked by NotI and EcoRI with N-terminal secretion BiP signal (MKLCILLAVVAFVGLSLG, SEQ ID NO: 1) and a double Strep-tag on C-terminus (WSHPQFEKGGGSGGGSGGSSAWSHPQFEK: SEQ ID NO: 2) was ordered from GeneArt with Drosophila codon optimization and cloned into the pExpres2-1 vector with a zeocin resistance gene (ExpreS2ion Biotechnologies, Denmark). This construct was called ‘hEPO, pExpres2-1’.

The Receptor Binding Domain comprising of aa319-591 of SARS-CoV-2 Spike 1 (Sequence ID: QIA20044.1) was designed as described by C. Fougeroux et al. (“Capsid-like particles decorated with the SARS-CoV-2 receptor-binding domain elicit strong virus neutralization activity,” Nat. Commun., vol. 12, no. 1, pp. 1-11, 2021, doi: 10.1038/s41467-020-20251-8) with a BiP secretion signal and Catcher at the N terminus and C-tag (EPEA) at the C terminus. The construct was codon optimized for expression in Drosophila and flanked by EcoRI and NotI restriction sited for cloning into pExpreS2-2 vector with geneticin selection marker.

### S2 Cell Handling, Transfection, and Cloning

Wild type Drosophila S2 cells were used as a starting cell line (ExpreS²ion Biotechnologies, Denmark) and stably transfected first with ‘XylT, pExpres2-2’, cloned and then the chosen clone was stably re-transfected with ‘hEPO, pExpres2-1’.

Stable transfections were made at the concentration of 2×10⁶ cells/ml in a volume of 5 ml of EX-CELL 420 serum free media (Sigma) supplemented with Pen/Strep (Sigma-Aldrich cat. no P4333-100M) in 25 ml tissue-culture flask. 50 μl of ExpreS2 Insect TRx5 transfection reagent is added, culture swirled and 12.5 ug of purified DNA is added, followed by swirling. A stable cell line was obtained after four weeks of selection with 4000 ug/ml Geneticin (G418, InvivoGen) or 1500 ug/ml zeocin (Thermo Fisher), respectively. Cloning of the cells was performed by limited dilution in 96 well plates with addition of feeder cells, 10% serum (Fetal Bovine Serum, Cat. no 10100-147, ThermoFisher) and G418 selection.

Production and Purification of hEPO and SARS-CoV-2 RBD

A clone confirmed to have the highest percentage of xylosylated glycans and transfected with strep tagged hEPO, pExpres2-1 was expanded to 0.5 l. Supernatant was harvested and filtered through a 0.22 μm cutoff filter. The supernatant was concentrated using tangential flow filtration (TFF) four times fold and buffer exchanged against one I of buffer W (100 mM Tris/HCl, 150 mM NaCl, 1 mM EDTA, pH 8.0) and eluted with buffer BXT (buffer W with 50 mM biotin) according to the manufacturer's instructions (Iba). For the production of RBD antigen, the clones chosen for the highest content of xylosylation, S2-Xyl clone 8 and the S2-WT, were transiently transfected with plasmid encoding RBD. The cells were harvested three days post transfection and the C-tagged protein was purified using CaptureSelect™ C-tagXL Pre-packed Column (Thermo Fisher, 494307201) with wash Buffer A (25 mM Tris, 100 mM NaCl, pH=7.5) and eluted with Buffer B (buffer A with 2M MgCl₂) with step elution. The supernatant was up-concentrated, and buffer exchanged 10× with buffer A prior to purification using TFF. Captured RBD was up-concentrated and monomeric RBD was separated using a Superdex-200 pg 26/600 (Cytvia) SEC column in 1×PBS.

### Anti-Xylose Western Blot

Samples from supernatant were analysed for xylose signal by Western blot. Briefly, samples were reduced and run on a 4-12% SDS-PAGE gel and transferred to a nitrocellulose membrane (Invitrogen™ IB301002). The membrane was blocked by casein-blocking buffer (Sigma) then incubated with primary rabbit polyclonal Anti β1,2-xylose antibody (Agrisera, AS07267, 1:5000), and detected by secondary HRP conjugated polyclonal goat anti-rabbit antibody (Dako Denmark, P0448). The signal was detected using the ECL Chemiluminescent Substrate Reagent Kit (Novex™, Thermo Fisher WP20005).

### Glycoprofiling

N-glycan analysis was performed as described in Link A. et al., J. Immunol., vol. 188, no. 8, pp. 3724-3733, 2012, DOI: 10.4049/jimmunol.1103312. N-glycans were released from purified proteins and fluorescently labelled with the GlycoPrep Rapid N-glycan Kit (ProZyme Inc.) or the GlycoWorks RapiFluor-MS N-glycan Kit (Waters). Labelled glycans were analysed by Liquid chromatography-mass spectrometry (LC-MS) on a Thermo Ultimate 3000 HPLC (fluorescence detector coupled to Thermo Velos Pro Ion Trap MS).

cVLPs Design, Expression, and Purification

CLPs were designed and purified as described in C. Fougeroux et al., Nat. Commun., vol. 12, no. 1, pp. 1-11, 2021, doi: 10.1038/s41467-020-20251-8. In brief, Acinetobacter phage AP205 coat protein (Gene ID: 956335) with a proprietary peptide-binding Tag and a GSGTAGGGSGS (SEQ ID NO: 2) linker at the N-terminus was inserted into the pET28a (+) vector (Novagen) and expressed in BL21 (DE3) E. coli cells (New England Biolabs).

### Coupling and Separation of RBD-cVLP Vaccines

RBD glycovariants were mixed with cVLPs in 1:1 molar ratio in coupling buffer (1.2 mM KH₂HPO₄, 8.1 mM Na₂HPO₄*H₂O, 136.9 mM NaCl, 2.7 mM KCl, 10 mM Tris, 200 mM sucrose, pH 8.5) and incubated at room temperature overnight. Coupled samples were then up-concentrated on 15 ml Amicon concentrators with 30 kDa MWCO and loaded onto a Superdex-200 pg 26/600 (Cytvia) SEC column and eluted in 1×PBS (Gibco) to separate coupled cVLPs from unbound protein. Fractions containing coupled RBD-cVLPs were up-concentrated again and the concentration of RBD-CLPs was measured by the Bradford reaction. Endotoxin levels were measured using the Endosafe-PTS Portable System with LAL cartidges (Charles River).

### Mice Immunizations

Mice studies were authorized by the Danish National Animal Experiments Inspectorate (Dyreforsøgstilsynet, licence number 2018-15-0201-01541) and conducted according to the guidelines. BALB/c mice of age 14-16 weeks (Janvier, Denmark) were immunized intramuscularly (in the thigh) with either 15 μg of soluble antigen glycovariant (RBD-WT or RBD-Xyl) formulated with Addavax™ adjuvant (vac-adx-10, InvivoGen) or with 1 μg of cVLP-displayed glycovariants (RBD-WT)-CVLP or (RBD-Xyl)-cVLP. That accounted to four groups with five mice per group immunized. The immunizations were done as a prime followed by a booster three weeks after. The serum samples were collected one week before the prime immunization (pre-bleed) and two weeks after each immunization as 1^{st} and 2nd bleed, respectively.

### Antibody Response Measured by ELISA

The antibody response in immunized mice was measured by ELISA. 96-well Nunc Maxisorp plates (Invitrogen, 44-2404-21) were coated with 50 μl 2 μg/ml of SARS-CoV-2 Spike Protein S1 (aa14-683, His-Avi Tag Recombinant Protein, Sigma RP-87681) produced in HEK293 cells and incubated at 4° C. over night. The next day plates were incubated with 200 μl of casein blocking solution (Merck, B6429-500ML) at room temperature for two hours. Sera were diluted 50× (for total IgG measurements) or 100× (for IgG subclasses measurement) and then further diluted three-fold in 1×PBS. 50 μl of dilutions were added per well and incubated for 1.5 hour at room temperature. Plates were then incubated with HRP-conjugated polyclonal goat anti-mouse antibody (Dako, P0447) in casein blocking solution for measurement of total IgG antibodies. For measurement of the IgG classes, the antibodies used were goat anti-mouse IgG1, IgG2a, IgG2b and IgG3 (Sigma, A10551, M32207, M32407, M32707, respectively). Plates were washed three times with 1×PBS with 0.05% Tween20 between all the steps. Plates were developed using 100 μl of TMB Xtnd substrate (Kem-En-Tec Nordic cat. no. 5280A) and the reaction was stopped with 100 μl of 0.35M sulfuric acid after 10 min. OD₄₅₀ was measured using a Biotek ELx808 plate reader.

### Results

### Establishment, Cloning and Glycoprofiling of the S2-XylT Cell Line

A polyclonal cell line named S2-Xyl expressing the β1,2-Xylosyltransferase from A. thaliana was established.

The cell line was cloned by serial dilution, and the total secretome of ten clones was analysed for the presence of xylose by Western blot using an anti β1,2-xylose antibody. All clones showed a signal, contrary to the wild type.

The secretome of three clones of the S2-xyIT cell line (named S2-Xyl clone 6, S2-Xyl clone 7 and S2-Xyl clone 8) were further analyzed by LS-MS to obtain detailed glycan profiles and were compared with the wild type glycoprofile, see FIGS. 5a-5e.

FIG. 6 shows the relative percentages of released N-glycans from all secreted proteins. S2-S2-Xyl clone 6 has shown around 56% xylosylated N-glycans, while S2-Xyl clone 7 showed 44% and S2-Xyl-clone 8 showed 58%.

Glycosylation Profile of Purified hEPO

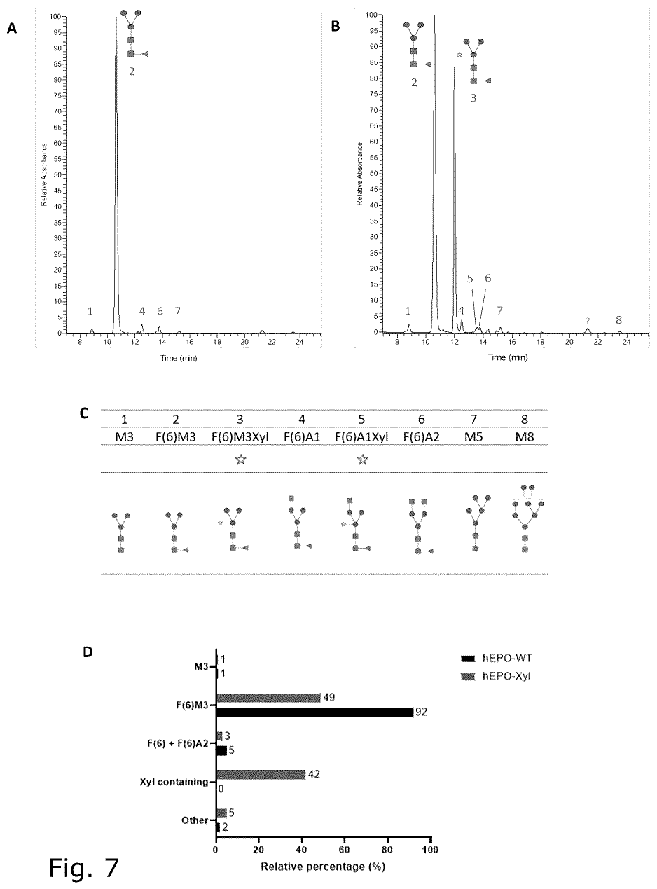

The xylosylation level was compared on purified protein level, to confirm establishment of a cell line stably expressing strep-tagged hEPO in the wild type S2 cells (S2-WT) and in the S2-Xyl clone with the high relative xylose content, S2-Xyl clone 8. Purified S2-WT hEPO showed one major F(6)M3 peak constituting around 92% of total area, which has shifted to 42% of xylose containing F(6)M3Xyl on hEPO from S2-Xyl clone 8, with a drop of F(6)M3 to 49%, FIG. 7 (A-C).

The comparison of relative abundance of N-glycans from hEPO-WT and hEPO-Xyl is shown in FIG. 7D.

### Analysis of Xylosylated N-Glycans on SARS-CoV-2 RBD Antigen

After establishment of the cell line with significant xylosylation level, the potential for increased immunogenicity of xylosylated glycans in vaccine formulations was investigated using RBD of Spike 1 protein of SARS-CoV-2 transiently expressed in S2-WT and in S2-Xyl clone 8 cell lines. The protein contains two glycosylation sites within the RBD part and two glycosylation sites in the Catcher part.

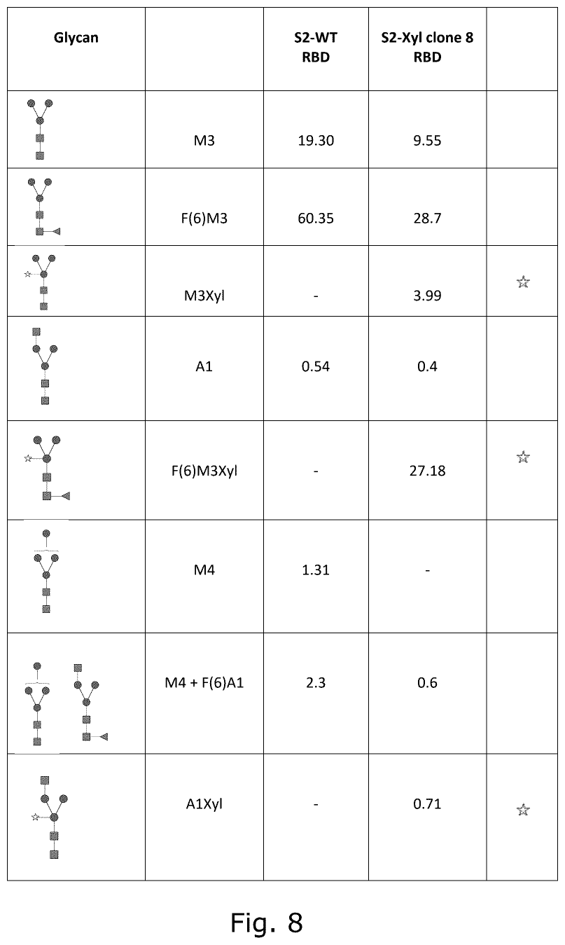

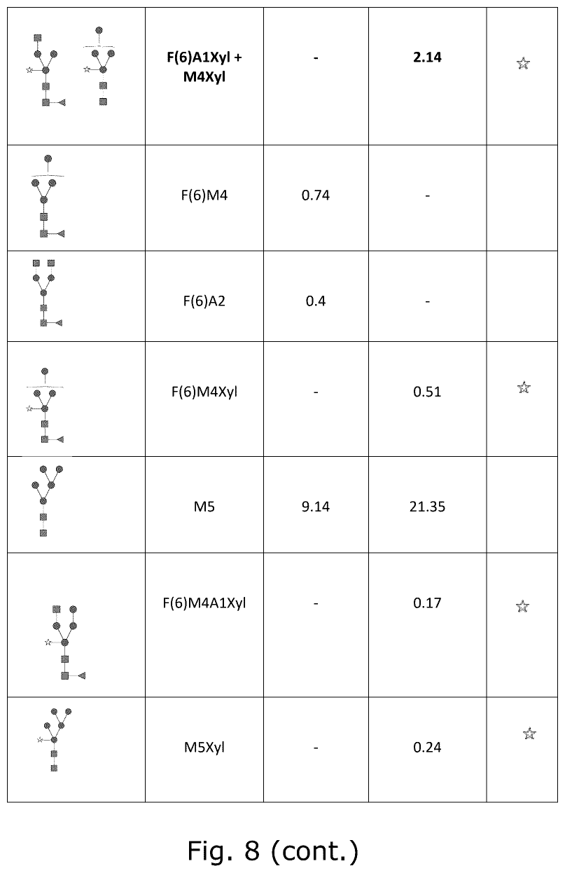

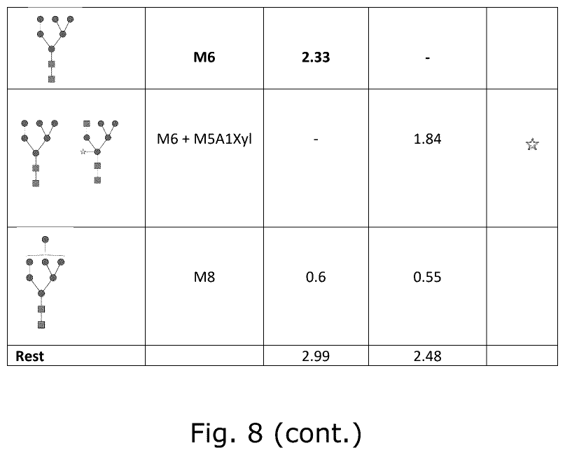

The RBD-WT glycosylation is not as homogenous as hEPO-WT glycosylation and has around 14% less of paucimannosidic glycans and while hEPO-WT has exclusively fucosylated paucimannose N-glycans, the RBD-WT has around 19% of non-fucosylated and 60% of fucosylated paucimannose glycans. All relative percentages are presented in FIG. 8.

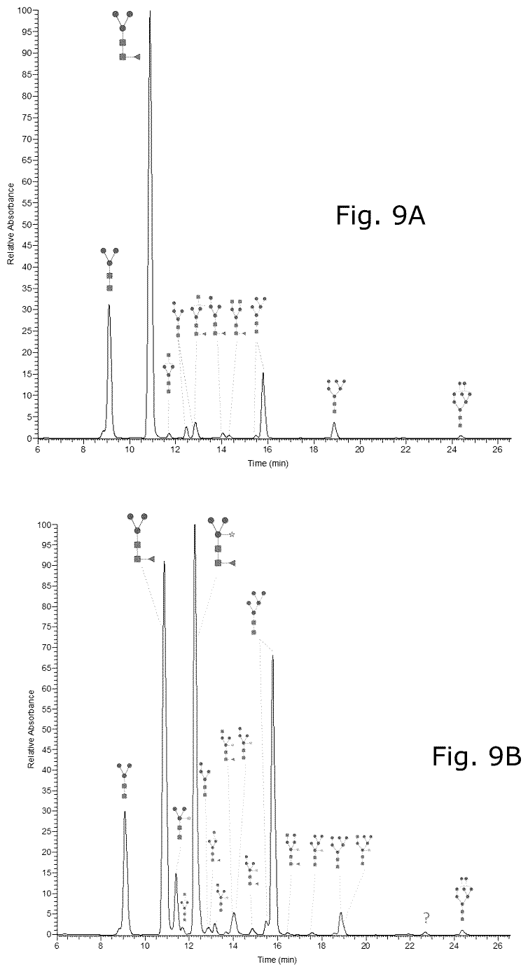

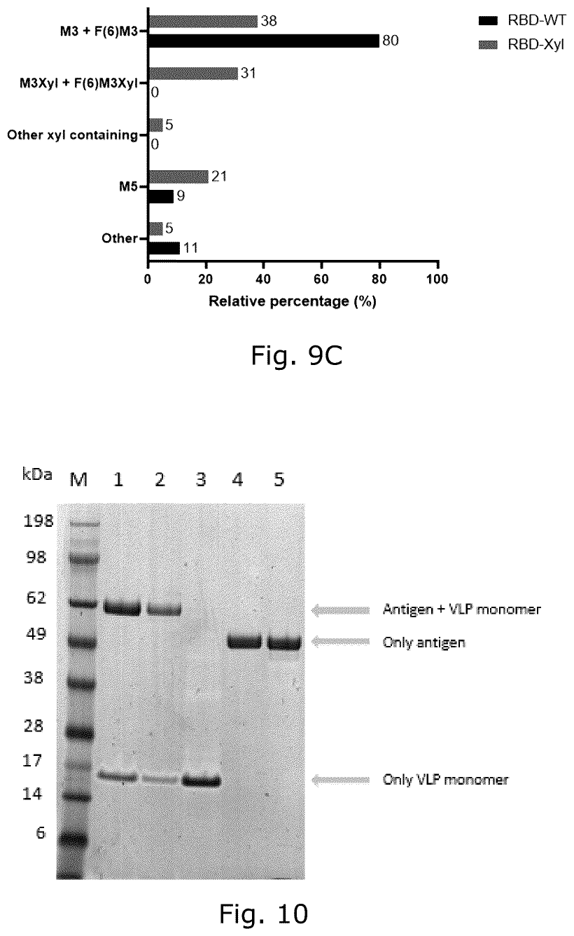

Interestingly, RBD-WT contains 9% of M5 glycans, which are at insignificantly low amounts in hEPO-WT, see FIG. 9A. RBD-Xyl released glycans showed around 31% of xylosylated paucimannose glycans and around 38% of non-fucosylated paucimannose glycans and around 21% of M5 glycans, see FIG. 9B. Other xylosylated glycan in RBD-Xyl contributed to total of around 36% of xylosylated glycans. The comparison of relative abundance of N-glycans from RBD-WT and RBD-Xyl is shown in FIG. 9C.

Coupling of SARS-CoV-2 RBD Antigen to cVLPs

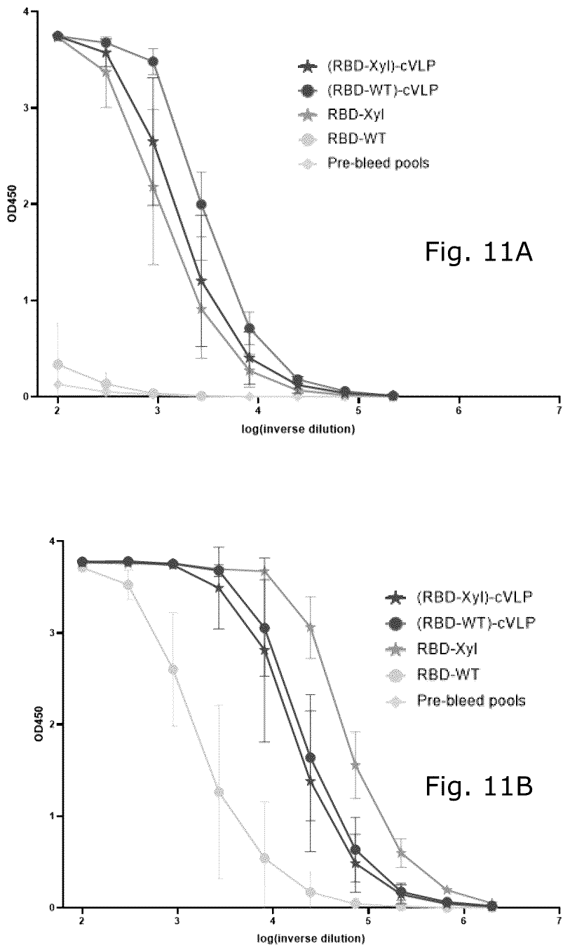

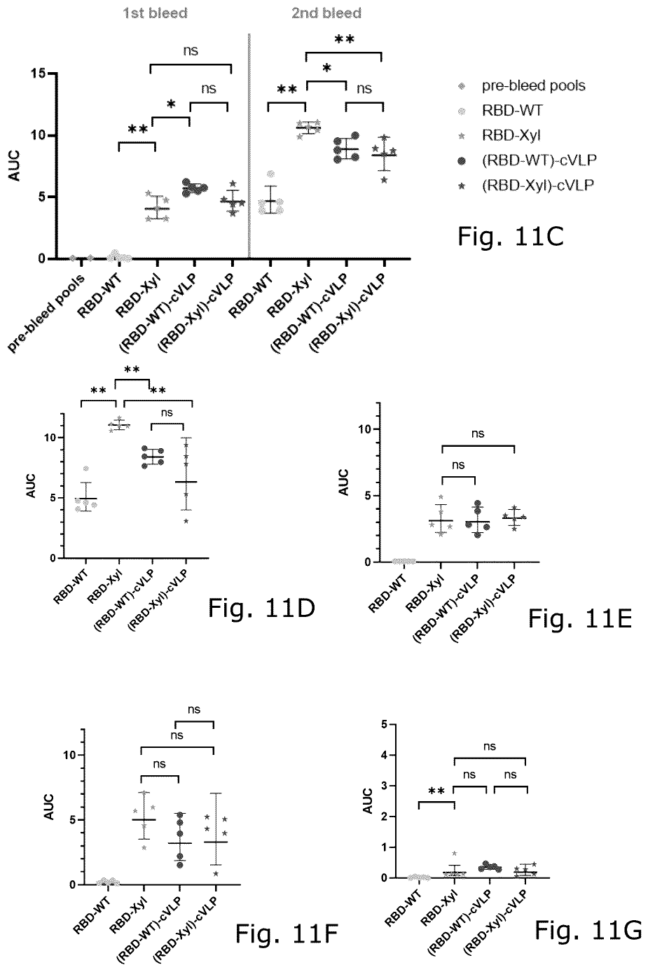

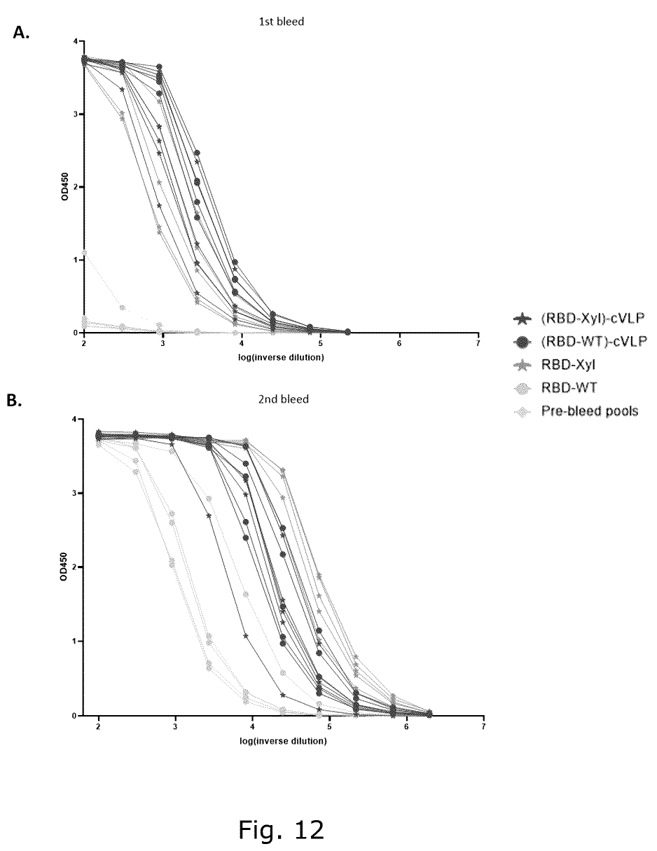

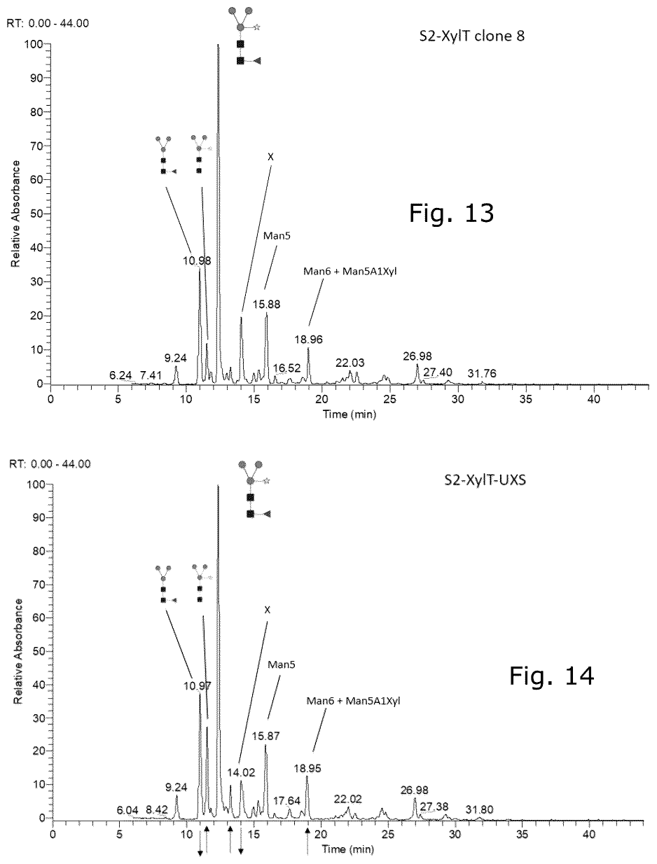

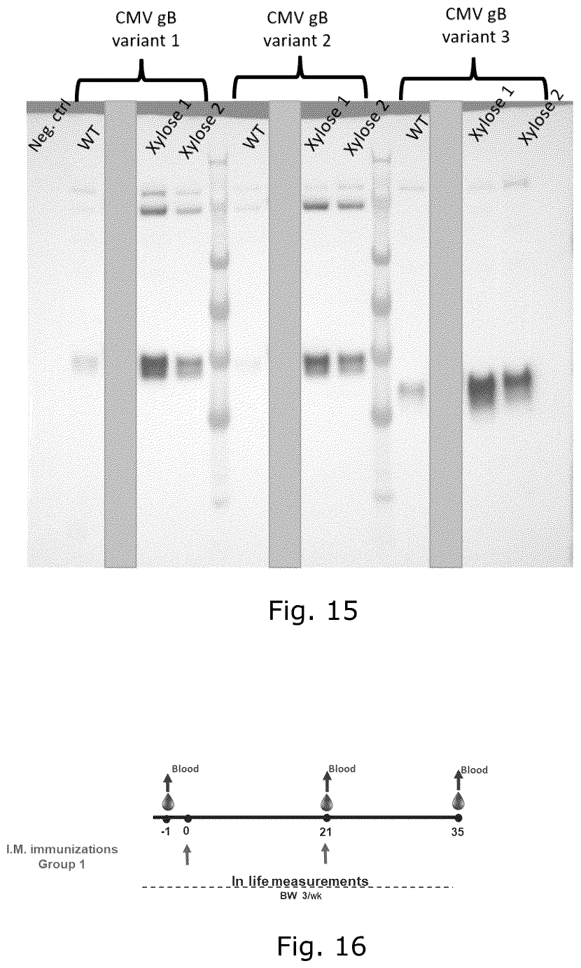

cVLP displayed vaccine formulations were made by mixing the antigen containing the Catcher with cVLPs containing the Tag in the coupling buffer. Excess of antigen was separated from coupled cVLPs size exclusion chromatography. Coupling of RBD antigens to cVLPs was confirmed by SDS-PAGE gel by the size change of cVLP-Tag monomer which is 16.5 kDa. The soluble antigen vaccines were called RBD-WT and RBD-Xyl and cVLP vaccines were called (RBD-WT)-CVLP and (RBD-Xyl)-cVLP. Reduced vaccine components seen on SDS-PAGE are presented on FIG. 10.

Mice Antibody Response to Immunization with RBD Glycovariants

The mice (n=5 in each group) were immunized with a three-week interval prime-boost regimen with four 15 μg soluble RBD glycovariants formulated with Addavax™ adjuvant (RBD-WT and RBD-Xyl) as well as cVLP-displayed forms without the adjuvant in 1 μg dose ((RBD-WT)-cVLP and (RBD-Xyl)-cVLP). The immunogenicity of the vaccines was examined by measuring total IgG and IgG subclasses response by ELISA towards the Spike 1 protein produced in human HEK293 cells. HEK293 produced protein was chosen as it was assumed that human cell line produced Spike 1 will be the closest to resemble the natural Spike 1 protein. FIGS. 12A and 12B depict total anti Spike 1 IgG titer dilution curves from four groups of vaccinations measured after 1^{st} and 2^{nd} vaccination. FIG. 12C depicts these dilution curves as Areas Under the Curve (AUCs). After the first vaccination, the RBD-WT elicited very little amounts of IgGs while the rest three vaccines elicited much higher levels. After the second vaccination, total IgG levels induced by RBD-WT went up to the level of other vaccines after the first vaccinations. The RBD-Xyl induced the highest level of IgGs, significantly higher than (RBD-WT)-CVLP (p=0.0159) and (RBD-Xyl)-cVLP (p=0.0079) and the difference between the cVLP-displayed glycovariants is not significant (p=0.0952). The IgG subclasses profiles were examined for four vaccine groups in sera after second vaccination and can be seen in FIGS. 12D-12G. The RBD-WT induced solely IgG1 subclass, while the other three vaccines showed IgG subclass switching to also IgG2a and IgG2b without significant difference in between them. The IgG1 subclass pattern for four vaccines looked similar to the total IgG measurement (cf. FIGS. 12C and 12D). For all dilution curves (and not geometric mean) at first and second bleed of total IgG antibodies see FIG. 12.

### Conclusions

RBD-Xyl is more immunogenic than RBD-WT in both types of formulations (free, soluble antigen and the VLP displayed formulation). Moreover, the soluble RBD-Xyl in the dosage 15 ug elicits more anti-Spike 1 antibodies than its VLP counterpart (Xyl-RBD)-VLP.

### Example 2

Establishment of a Further Cell Line (S2-XylT-UXS) Capable of Producing Proteins with Xylosylated N-Glycans

The “S2-XylT-UXS” cell line was established by transfecting the S2-XylT clone 8 from Example 1 with a plasmid comprising the Drosophila melanogaster UDP-xylose synthase gene (UXS, genomic sequence provided by Flybase ID: FBgn0035848, SEQ ID NO: 4, mRNA provided in SEQ ID NO: 5) and selecting for stable polyclonal cells. UDP-xylose synthase (SEQ ID NO: 6) is an enzyme that converts UDP-glucuronic acid into UDP-D-Xylose. The rationale behind expressing this gene was to potentially increase the UDP-D-Xylose cellular pool available for incorporation into N-glycans and eventually increase the xylosylation level of N-glycans in glycoengineered S2 cells.

The LS-MS analysis of released and labelled N-glycans was made to compare S2-XylT clone 8 (also termed “Xylose1” herein) and S2-XylT-UXS cell lines and can be seen in FIGS. 13 and 14.

A comparison of the N-glycosylation profiles shown in FIGS. 13 and 14 for secreted proteins demonstrate that the S2-XylT-UXS S2 cell line exhibits increased xylosylation compared to the S2 cell line from Example 1.

Further, three new vaccine targets, all variants of cytomegalovirus (CMV) glycoprotein B (Gb), were expressed in S2-XylT clone 8 and S2-XylT-UXS cell lines and showed slightly lower electrophoretic mobility in comparison to the wild-type S2 cells expressed and otherwise identical proteins. Variant 2 and variant 3 also seem to show higher apparent mass when produced in S2-XylT-UXS than S2-XylT clone 8, thus suggesting a higher xylose content. See FIG. 15.

### Example 3

Further Testing of Expression Products from Genetically Modified Cell Lines

### Candidate Vaccine Proteins

Several proteins will be compared with regards to immunogenicity in S2, High mannose cell lines (S2 cell lines produced high mannose protein as disclosed in WO 2020/144358), S2-XylT clone 8 and S2-XylT-UXS cell lines. Results are expected to verify increased immunogenicity for full length trimerized Hemagglutinin Influenza A virus (HA) protein for strains H1N1, H5N1 and H7N9, and for H5N1 HA stem only. Further tested protein will be influenza NP protein as well as M2 protein (strain: Brisbane, PR_8). In addition, Nipah virus G protein is tested for increased immunogenicity when produced in glycomodified cell lines compared to production in wildtype S2.

### Testing Glycomodified Proteins In Vivo

Glycomodified proteins will be purified by C-tag affinity chromatography and polished by gel filtration chromatography. Concentrations will be measured by OD₂₈₀ or Bradford assay. The level of endotoxin will be measured, and endotoxin will be removed if necessary.

In vivo testing will be done in rodents (mice, CD1) according to the schedule set forth in FIG. 16 and the following table for each of 3 glycomodified cell lines and a wild-type S2 cell line. Hence a total of 140 mice (4×35) will be immunized. Administration will be i.m with 100 μl per immunization (administered as 2×50 μl in the thighs). Blood will be sampled pre-immunization and as shown i in FIG. 16.

| Group | Treatment | Dose (μg) | Mice (#) |
|---|---|---|---|
| 1 | HA (H1N1) + adjuvant | 20 | 5 |
| 2 | HA (H5N1) + adjuvant | 20 | 5 |
| 3 | HA (H7N9) + adjuvant | 20 | 5 |
| 4 | HA (H5N1) HA stem only + adjuvant | 20 | 5 |
| 5 | Influenza NP + adjuvant | 20 | 5 |
| 6 | Influenze M2 + adjuvant | 20 | 5 |
| 7 | Nipah virus protein G + adjuvant | 20 | 5 |

All protein will be formulated in 1×PBS in admixture with AddaVax™ adjuvant.

Read-outs: Body weight (BW) change from first immunization and antigen-specific antibody titres via ELISA.

### EXPECTED RESULTS

The purified new glycoproteins will be used for mice immunizations to confirm xylosylation influence on vaccine potency on multiple targets as well as investigating potential advantages of vaccine candidates produced in the S2-XylT-UXS cell line over S2-XylT clone 8 cell line regarding higher immunogenicity caused by higher xylosylation. Immunogenicity will be determined by analysing mouse blood and quantify the polyclonal response via ELISA or AlphaLisa.

1. A non-plant polypeptide or protein comprising xylosylated N-linked glycans that comprise β1,2-xylose, wherein at least 25% of the individual protein species comprise xylosylated N-glycans, wherein said polypeptide or protein preferably is selected from the group consisting of VAR2CA, HER2 and hEPO; a viral protein or polypeptide from HIV, Ebola virus, Zika virus, Chikungunya virus, Dengue virus, Hepatitis A virus, influenza virus, Polio virus, Rabies virus, Measles virus, mumps virus, rubella virus, Rotavirus virus, Smallpox virus, Chickenpox virus, Hepatitis B virus, human papillomavirus, varicella zoster virus, Yellow fever virus, SARS-CoV-1, cytomegalovirus (CMV), and SARS-CoV-2; and a bacterial protein or polypeptide from Clostridium tetanii, Corynebacterium diphtheria, Haemophilus influenzae, Bordetella pertussis, Streptococcus pneumoniae, and Neisseria meningitides.

2. The non-plant polypeptide or protein according to claim 1, wherein the protein comprises F(6)M3, F(6)A1, F(6)A2 and/or F(3)F(6)M3 glycan structures.

3. The non-plant polypeptide or protein according to claims 1-2, further comprising increased Man-5-Man9 structures compared to the wild-type protein.

4. The non-plant polypeptide or protein according to any one of the preceding claims, which of mammalian, crustacean, insect, arachnoid, viral, bacterial, fungal, helminthic, or protozoan origin.

5. The non-plant polypeptide or protein according to any one of the preceding claims, wherein at least 25% of individual protein/peptide species comprise xylosylated N-glycans, such as at least 35%, at least 40%, at least 45%, at least 50%, at least 55%, at least 60%, at least 65%, at least 70%, 75%, and at least 90%.

6. The non-plant polypeptide or protein according to any one of the preceding claims, which is obtainable by a method comprising expressing polynucleotide(s) encoding the polypeptide or protein in an S2 cell genetically altered so as to produce an active β1,2-xylosyltransferase and optionally an active UDP-xylose synthase or an increased amount of active UDP-xylose synthase.

7. The non-plant polypeptide or protein according to any one of the preceding claims, further comprising α1,3-linked fucose.

8. The non-plant polypeptide or protein according to any one of the preceding claims, which further comprises or is coupled via a non-peptide bond to a heterologous moiety, such as a purification tag, an immunogenic carrier molecule or T-helper lymphocyte epitope, a solubility-modifying group, a protraction group, a targeting moiety, a virus-like particle, and an immune modulating moiety, said heterologous moiety optionally being fused to the polypeptide or protein via a peptide linker.

9. An immunogenic composition comprising the non-plant polypeptide or protein according to any one of the preceding claims in admixture with at least one immunological adjuvant and optionally a pharmaceutically acceptable carrier and/or diluent and/or excipient.

10. The immunogenic composition according to claim 9, which is preferably in the form of a liquid formulation such as a solution, a suspension, an emulsion, or a suspoemulsion, or in the form of a solid or semisolid formulation, such as a powder, tablet, suppository, pill, gel, cream, or ointment.

11. The immunogenic composition according to claim 9 or 10, wherein the immunological adjuvant is selected from the group consisting of an aluminium salt, an oil-in-water emulsion, a saponin, complete and incomplete Freund's adjuvant, and a cytokine.

12. The immunogenic composition according to claim 11, wherein the emulsion is a squalene-based oil-in-water emulsion.

13. The immunogenic composition according to any one of claims 9-12, which is contained in a unit dose form, such as in freeze-dried form.

14. The non-plant polypeptide or protein according to any one of claims 1-8 or the composition according to any one of claims 9-13, or a non-plant polypeptide or protein comprising xylosylated N-linked glycans that comprise β1,2-xylose, wherein at least 25% of the individual protein species comprise xylosylated N-glycans for use in a method for inducing or enhancing a specific immune response in an animal, such as a human being, the method comprising at least one immunization of the animal with an effective amount of the non-plant polypeptide or protein comprising N-linked glycans that comprise β1,2-xylose, the protein or polypeptide according to any one of claims 1-8 or the composition according to any one of claims 9-13, wherein the method is for disease prophylaxis or for treatment or amelioration of disease such as SARS-CoV-2, influenza, pr CMV infection; and wherein a priming immunization and at least one booster immunization is preferably administered.

15. The non-plant polypeptide, protein or composition for use according to claim 14, further comprising N-linked glycans that comprise α1,3-linked fucose, wherein at least 25% of individual protein/peptide species comprise α1,3-linked fucose.

16. The non-plant protein, polypeptide or composition for the use according to claim 14 or 15, wherein the at least one immunization reduces risk in the animal of contracting a disease caused by an infectious organism or where the immunization modulates an existing immune response against the protein or polypeptide, or wherein the at least one immunization treats or ameliorates or reduces risk of disease caused by or associated with an autologous protein or by a cell producing said autologous protein.

17. A genetically modified non-plant eukaryotic cell, such as a mammalian cell or an insect cell or a fungal cell such as a yeast, which comprises at least one heterologous polynucleotide sequence encoding and expressing β1,2-xylosyltransferase, wherein the cell is capable of producing N-glycosylated protein carrying β1,2-xylose groups, and where the heterologous polynucleotide sequence preferably is the XylT gene from Arabidobsis thaliana, or an equivalent polynucleotide encoding a plant β1,2-xylosyltransferase.

18. The genetically modified non-plant eukaryotic cell according to claim 17, which further comprises at least one polynucleotide sequence encoding and expressing a UDP-xylose synthase or comprises at least one additional UDP-xylose synthase encoding sequence so as to exhibit increased UDP-xylose synthase activity.

19. The genetically modified non-plant eukaryotic cell according to claim 18, wherein the UDP-xylose synthase has the amino acid sequence SEQ ID NO: 6, which is optionally encoded by SEQ ID NO: 5.

20. The genetically modified non-plant eukaryotic cell according to any one of claims 17-19, further comprising at least one heterologous polynucleotide sequence encoding and expressing a heterologous α1,3-fucosyltransferase, and wherein expression of the α-Man-Ia gene optionally has been reduced or abolished or wherein expression of genes encoding enzymes extending glycans beyond Man3, such as beyond Man5 optionally has been increased, wherein the cell is capable of producing N-glycosylated protein carrying α1,3-fucosyl groups, and which preferably exhibits reduced or abolished function of at least one α1,6-fucosyltransferase encoding gene, and where the heterologous polynucleotide sequence preferably is the fuc11 gene from Arabidobsis thaliana, or an equivalent polynucleotide encoding a plant α1,3-fucosyltransferase.

21. The genetically modified cell according to any one of claims 17-20, which further expresses a heterologous gene encoding a polypeptide or protein according to any one of claims 1-8, and wherein >25% of said polypeptide or protein preferably displays β1,2-xylose glycans, such as >30%, >35%, >40%, >45%, >50%, >55%, >60%, >65%, >70%, >75% and more than 85% of said polypeptide or protein.

22. The genetically modified cell according to any one of claims 17-21, which is an insect cell, preferably a Drosophila melanogaster cell, such as an S2 or S3, or another insect cell such as Sf9, SF21, High5, and C6-36, or which is a mammalian cell such as a CHO or HEK.

23. A cell line, such as a clonal cell line, comprising the genetically modified cell according to any one of claims 17-22.

24. A method for producing an N-glycosylated polypeptide or protein carrying β1,2-xylose, the method comprising culturing a cell according to any one of claims 17-22 or a cell line according to claim 23, wherein the cell line expresses a polynucleotide encoding the amino acid sequence of the N-glycosylated polypeptide or protein, and subsequently isolating the N-glycosylated polypeptide or protein from the culture mixture, and optionally subjecting the N-glycosylated polypeptide or protein to further purification.
